(19) United States
(12) Patent Application Publication
(10) Pub. No.: US 2026/0271853 A1
(43) Pub. Date: Sep. 17, 2026

(54) MODIFIED NUCLEIC ACID SEQUENCE FOR CONFERRING CLUBROOT RESISTANCE TO CANOLA AND METHODS OF PRODUCING SAME

(71) Applicant: THE GOVERNORS OF THE UNIVERSITY OF ALBERTA, Edmonton (CA)

(72) Inventors: Habibur RAHMAN, Edmonton (CA); Kawalpreet KAUR, Edmonton (CA)

(21) Appl. No.: 19/162,601

(22) PCT Filed: Mar. 7, 2024
(86) PCT No.: PCT/CA2024/050281

§ 371 Date: Sep. 5, 2025

Related U.S. Application Data
(60) Provisional application No. 63/488,824, filed on Mar. 7, 2023.

(51) Int. Cl. A01H 6/20; A01H 1/00; A01H 1/04; C12N 5/04

(52) U.S. Cl. CPC A01H 6/20; A01H 1/045; A01H 1/1245; C12N 5/04

(57) ABSTRACT

Embodiments herein are related generally to disease resistance genes in plants and, specifically, to a modified nucleic acid sequence for conferring clubroot disease resistance in canola and methods of producing same.

### CROSS REFERENCE TO RELATED APPLICATIONS

This application claims priority to U.S. Patent Provisional Application 63/488,824, filed Mar. 7, 2023.

### FIELD

Embodiments herein are related generally to disease resistance genes in plants and, specifically, to a modified nucleic acid sequence for conferring clubroot disease resistance in canola and methods of producing same.

### BACKGROUND

Clubroot is a soil-borne disease caused by the pathogen Plasmodiophora brassicae (P. brassicae). It is a major threat to oilseed and vegetable crop production worldwide, specifically affecting plants in the Brassica genus such as Brassica napus (B. napus) including canola, and Brassica rapa (B. rapa) including turnip and Chinese cabbage, and Brassica oleracea (B. oleracea)(Dixon 2009). This disease results deformed club shaped roots or ‘galls,’ and the clubbed roots inhibit the uptake of water and nutrients and result in stunted growth, wilting, chlorosis, and even death of the plant (Kageyama and Asano 2009). In the case of canola, this disease causes about 30% yield loss; however, in extreme case, complete failure of the crop has been reported (Tewari et al. 2005; Howard et al. 2011). Aside from yield loss, this disease can result in 2%-6% decreased seed oil content (Engqvist 1994; Pageau et al. 2006) and 50% increased chlorophyll content in oil (Engqvist 1994), and thereby negatively affects the seed quality traits.

The resting spores of P. brassicae can persist in soil more than 17 years (Wallenhammar 1996); therefore, eradication of this pathogen from soil is extremely difficult once it is established in soil. Further, the structure of the pathotype population of P. brassicae in soil can change in a short period of time (Strelkov et al. 2016). For example, in the initial stage of the occurrence of clubroot disease in Canada, P. brassicae pathotype 3 or 3H was the most virulent and prevalent. However, new virulent pathotypes, such as 3A, 2B, 3D, and 5X evolved in canola fields after growing the resistant-categorized cultivars for about 10 years, rendering some of the known resistances ineffective (Strelkov et al. 2016; Strelkov et al. 2018). It has been reported that 3H was the most common pathotype in the Peace Country of Canada in 2017; however, new pathotypes such as 8C were detected in this region in 2018 (Strelkov et al. 2021). As such, control of this disease through known cultural and crop management practices is not possible. The development of clubroot resistant cultivars is essentially needed to protect the crop from this disease (Hasan et al. 2021).

To date, more than 20 major and minor gene loci conferring resistance to different P. brassicae pathotypes have been reported on the ‘A’ chromosomes of B. napus and B. rapa, including A01, A02, A03, A05, A06, A07 and A08. Among the ‘A’ genome resistances, the resistance genes located on A03 and A08 exhibit major and dominant effect; therefore, these resistances have been used extensively by breeders and researchers to develop clubroot resistant Brassica crop cultivars including canola cultivars in different countries including Canada (Rahman et al. 2014; Hasan et al. 2021). In contrast to B. napus and B. rapa, clubroot resistance in the ‘C’ chromosomes of Brassica oleracea (B. oleracea) is often under polygenic control with the involvement of recessive to dominant gene loci with a small to large effect (Hasan et al. 2021; Farid et al. 2020). This tendency complicates the use of B. oleracea as a source for resistance genes and, instead, known B. napus and B. rapa lines are the preferred sources of resistance genes given their relative ease of study and characterization. Lines of B. rapa are especially promising, as is described in more detail, below.

In Canada, the major clubroot resistance genes of A03 were obtained from different sources, such as the European winter oilseed B. napus cv. ‘Mendel’ (Rahman et al. 2011), B. rapa subsp. chinensis cv. Flower Navina (Chu et al. 2013) and Chinese cabbage (B. rapa ssp. pekinensis) (Hirani et al. 2016; Hasan et al. 2021). Among these, resistance of the cv. ‘Mendel’ has been used extensively in breeding, and this resistance is derived from a turnip (B. rapa ssp. rapifera, AA, 2n=20) accession ‘ECD 04’ (Fredua-Agyeman et al. 2016). In case of A08, the major resistance has been introgressed from various sources including the rutabaga cv. Brookfield (Hasan and Rahman 2016) and Polycross (Wang et al. 2022), of which the Brookfield-resistance has been used to develop canola cultivars. Recent study demonstrated that the resistance located on A03 and A08 chromosomes of the rutabaga cv. Polycross exhibit non-allelic interaction and contribute to the resistance (Wang et al. 2022).

To date, several clubroot resistant canola cultivars have been developed by using a single clubroot resistance gene of A03 or A08. However, a cultivar carrying a single major clubroot resistance gene has been reported to become ineffective after a few years of cultivation (Hirai 2006; Strelkov et al. 2016). This is primarily due to the change in population structure of the pathogen P. brassicae in soil as well as evolution of new virulent pathotypes such as pathotype 3A, 2B, 3D and 5X (Strelkov et al. 2018, 2021). Therefore, introgression of additional major genes into canola is needed to broaden the genetic base of clubroot resistance genes in this crop for resistance to multiple pathotypes including the recently evolved ones. Also, the availability of multiple resistance genes will enable canola breeders and researchers to pyramid more than one resistance gene in a cultivar. Pyramiding of multiple resistance genes or major genes conferring resistance to various pathotypes, or a combination of major genes with minor genes is thought to increase the durability of resistance (Boskovic et al. 2001; Werner et al. 2005; Ellis et al. 2014; Fukuoka et al. 2015; Pilet-Nayel et al. 2017). The accumulated genes may impart resistance to either multiple pathotypes of a pathogen or provide high level resistance to a single pathotype (Matsumoto et al. 2012; Tomita et al. 2013; Shah et al. 2019). It is known that pyramiding of three major clubroot resistance genes, CRa, CRk and CRc, in a Chinese cabbage line gave resistance to six isolates of P. brassicae. Similarly, the pyramiding of a major gene and a minor gene has been shown to increase resistance to multiple pathotypes in B. napus (Shah et al. 2019) and B. oleracea (Tomita et al. 2013). The approach of gene pyramiding for resistance to multiple pathotypes has been considered a strategy for the improvement of disease resistance in other crops, such as for rust (leaf, stem, and strip) and fusarium head blight resistance in wheat (Boskovic et al. 2001; Laroche et al. 2019), barley stem rust (Sharma Poudel et al. 2018) and barley yellow mosaic virus complex resistance (Werner et al. 2005), and blast resistance in rice (Fukuoka et al. 2015; Jiang et al. 2019).

There remains a need for new canola plants carrying one or more than one clubroot resistance gene to combat rapidly changing P. brassicae pathotypes in the field.

### SUMMARY

In one aspect there is provided an isolated Plasmodiophora brassicae (P. brassicae) resistant Brassica plant cell comprising in its genome an A09 resistance locus comprising the interval from about 28.7 to about 41.5 Mb of A09.

In one example, wherein the A09 resistance locus is in the interval from about 38.01-41.5 Mb of A09

In one example, wherein the A09 resistance locus is in the interval from about 28.7-37.3 Mb of A09.

In one example, wherein the A09 resistance locus is at about 28.7-37.3 Mb of A09.

In one example, further comprising in its genome one or more of:
an A03 resistance locus comprising the interval from about 1.7 to about 14.5 Mb of A03,
an A02 resistance locus comprising the interval from about 1 to about 4 Mb of A02, or
an A08 resistance locus comprising the interval from about 14.34 Mb to about 20.87 Mb of A08.

In one example, wherein the A03 resistance locus is in the interval of about 1.7-10.2 Mb of A03.

In one example, wherein the A03 resistance locus is in the interval of about −10.9-14.5 Mb of A03.

In one example, wherein said plant cell is heterozygous for said resistance locus.

In one example, wherein said plant cell is homozygous for said resistance locus.

In one example, wherein said plant cell is resistant to one or more of to one or more of P. brassicae pathotypes 3H, 2B, 3A, 3D, 5G, 5X, 5L, 5O, 8E, or 8P.

In one example, wherein the Brassica plant cell is Brassica napus, Brassica rapa, or Brassica oleracea.

In one aspect there is provided A Seeds, fruit and/or plant part of comprising a Brassica plant cell as claimed in any one of claims 1 to 11.

In one aspect there is provided a method for producing a clubroot resistant Brassica plant, said method comprising:
identifying at least one Brassica plant comprising in its genome an A09 resistance locus comprising the interval from about 28.7 to about 41.5 Mb of A09, and
selecting a plant comprising said A09 resistance locus comprising the interval from about 28.7 to about 41.5 Mb of A09.

In one example, wherein the A09 resistance locus is in the interval from about 38.01-41.5 Mb of A09.

In one example, wherein the A09 resistance locus is in the interval from about 28.7-37.3 Mb of A09.

In one example, wherein the A09 resistance locus is at about 28.7-37.3 Mb of A09.

In one example, further comprising identifying in its genome one or more of:
an A03 resistance locus comprising the interval from about 1.7 to about 14.5 Mb of A03,
an A02 resistance locus comprising the interval from about 1 to about 4 Mb of A02, or
an A08 resistance locus comprising the interval from about 14.34 Mb to about 20.87 Mb of A08, and
selecting a plant comprising said one or more resistance locus.

In one example, wherein the A03 resistance locus is in the interval of about 1.7-10.2 Mb of A03.

In one example, wherein the A03 resistance locus is in the interval of about −10.9-14.5 Mb of A03.

In one example, wherein said plant is heterozygous for said resistance locus.

In one example, wherein said plant is homozygous for said resistance locus.

In one example, wherein said plant is resistant to one or more of to one or more of P. brassicae pathotypes 3H, 2B, 3A, 3D, 5G, 5X, 5L, 5O, 8E, or 8P.

In one aspect there is provided a method for providing a Plasmodiophora brassicae—(P. brassicae) resistant plant according to any one of claims 1 to 11 introgression or genomic combination.

In one aspect there is provided a Plasmodiophora brassicae—(P. brassicae) resistant Brassica plant comprising in its genome an A09 resistance locus comprising the interval from about 28.7 to about 41.5 Mb of A09.

In one example, wherein the A09 resistance locus is in the interval from about 38.01-41.5 Mb of A09

In one example, wherein the A09 resistance locus is in the interval from about 28.7-37.3 Mb of A09.

In one example, wherein the A09 resistance locus is at about 28.7-37.3 Mb of A09.

In one example, further comprising in its genome one or more of:
an A03 resistance locus comprising the interval from about 1.7 to about 14.5 Mb of A03,
an A02 resistance locus comprising the interval from about 1 to about 4 Mb of A02, or
an A08 resistance locus comprising the interval from about 14.34 Mb to about 20.87 Mb of A08.

In one example, wherein the A03 resistance locus is in the interval of about 1.7-10.2 Mb of A03.

In one example, wherein the A03 resistance locus is in the interval of about −10.9-14.5 Mb of A03.

In one example, wherein said plant is heterozygous for said resistance locus.

In one example, wherein said plant is homozygous for said resistance locus.

In one example, wherein said plant is resistant to one or more of to one or more of P. brassicae pathotypes 3H, 2B, 3A, 3D, 5G, 5X, 5L, 5O, 8E, or 8P.

In one example, wherein the Brassica cell plant is Brassica napus, Brassica rapa, or Brassica oleracea.

### BRIEF DESCRIPTION OF THE FIGURES

Embodiments of the present disclosure will now be described, by way of example only, with reference to the attached Figures.

FIG. 1 is a visual representation of the presently disclosed modified nucleic acid sequence, according to embodiments. The modified nucleic acid sequence comprises a region derived from chromosome A09 of canola and, optionally, at least one additional region derived from chromosomes A02, A03, and/or A08 of canola.

FIG. 2 is a set of bar charts showing frequency distributions of single nucleotide polymorphisms (SNPs) on Brassica napus (B. napus) chromosomes A01-A10 based on genome assembly of B. napus cv. Darmor-bzh v4.1 and B. rapa cv. Chiifu-401 v3.0. The numbers shown above each bar indicate the percent of total SNPs localized to each chromosome.

FIG. 3 is a set of bar charts showing frequency distributions of SNPs from the chromosomes A03 and A08 identified based on the ΔSNP index, which is an indication of the SNPs to be associated with resistance to P. brassicae pathotype 3H. ΔSNP index was calculated based on SNP data obtained by using the B. napus cv. Darmor-bzh v 4.1 and B. rapa cv. Chiifu-401 v3.0 genome assemblies.

FIG. 4 is a set of bar charts showing frequency distributions of the SNPs from the chromosomes A02, A03, A08, and A09 identified based on the ΔSNP index, which is an indication of the SNPs to be associated with resistance to P. brassicae pathotype 3A. ΔSNP index was calculated based on SNP data obtained by using the B. napus cv. Darmor-bzh v 4.1 and B. rapa cv. Chiifu-401 v3.0 genome assemblies.

FIG. 5 is a set of physical (left) and genetic (right) linkage maps of the Brassica A genome chromosomes A02, A03, A08, and A09. Physical maps were constructed based on position of the markers on B. rapa cv. Chiifu-401 v3.0 genome assembly and presented as million base pairs (Mb), and the genetic map as constructed using software JoinMap 4.1 (Van Ooijen 2011) and presented as centimorgan (cM). The position of the clubroot resistance loci on the linkage maps are shown with black solid lines adjacent to the markers. In case of position on the physical maps, the gray color within the chromosome indicates the genomic region associated with resistance to P. brassicae pathotype 3A identified through quantitative trait loci (QTL) mapping; the star and delta adjacent to the markers indicates the genomic regions associated with resistance that were identified through single marker analysis (SMA) and genome-wide association study (GWAS) approaches, respectively.

FIG. 6 depicts an agarose gel picture of 92 Brassica napus recombinant-inbred lines (RIL) lines genotyped with simple sequence repeat (SSR) markers SSR-5735 from A09 (37.7 Mb region) and SSR-6123 (1.6 Mb region) from A03 QTL; Invitrogen 100 bp Ladder (Thermo Fisher) is also included in the gel picture. The band of about 200 bp size, generated by SSR-5735 marker of the A09 locus, and the band of about 290 bp size, generated by SSR-6123 marker of the A03 locus, showed co-segregation with clubroot resistance. RP signifies the resistant parent ECD 01 (Brassica rapa var. rapifera); SP signifies the susceptible parent B. napus spring canola line A04-73NA; R and S signifies the resistant and susceptible lines of the RIL population, respectively.

FIG. 7 depicts Manhattan and Q-Q plot analysis using 92 Brassica napus recombinant inbred lines (RIL) derived from (B. napus x B. rapa turnip cv. Debra ECD 01)×B. napus interspecific cross for resistance to Plasmodiophora brassicae pathotype 3A. a) Manhattan plot; x-axis represents the 19 B. napus chromosomes and y-axis represents LOD [−log (P-value)] of the SNPs. b) Q-Q plot; x-axis represents the expected LOD [−log(P-value)] and the y-axis represents the observed LOD [−log(P-value] for the SNPs.

FIG. 8 depicts Venn diagrams showing differentially expressed genes (DEGs) (p-value ≥0.05) in clubroot resistant and susceptible Brassica napus near isogenic lines (NILs) due to infection by Plasmodiophora brassicae pathotype 3H at (a) 7 days after inoculation (dai) and (b) 14 dai from three comparisons of the resistant (R) and susceptible (S) near-isogenic lines (NILs) with or without inoculation. The overlapping regions shows the number of DEGs detected from more than one comparison. (c) Histogram of the number of upregulated and downregulated DEGs detected at 7- and 14 dai from three comparisons: Resistant inoculated (RI) NILs against its control (RC), susceptible inoculated (SI) NILs against its control (SC), and resistant inoculated (RI) NILs against susceptible inoculated (SI) NILs.

FIG. 9 depicts Venn diagrams representing the number of upregulated and downregulated differentially expressed genes (DEGs) in Brassica napus near isogenic lines (NILs) due to infection by Plasmodiophora brassicae pathotype 3H at 7 days after inoculation (dai) and 14 days after inoculation (dai) from three comparisons of the near-isogenic lines (NILs): (a) resistant inoculated (RI) NILs against its control (RC), (b) susceptible inoculated (SI) NILs against its control (SC), and (c) resistant inoculated (RI) NILs against susceptible inoculated (SI) NILs.

FIG. 10 depicts heatmaps representing the expression profiles of the differentially expressed genes (DEGs) related to different biological pathways in the clubroot resistant and susceptible Brassica napus near isogenic lines (NILs) (CR-NILs and CS-NILs) inoculated with Plasmodiophora brassicae pathotype 3H with respect to their controls at 7- and 14-days after inoculation (dai). RI=inoculated resistant NILs; RC=uninoculated (control) resistant NILs; SI=inoculated susceptible NILs; SC=uninoculated (control) susceptible NILs.

FIG. 11 depicts Heatmaps representing the expression profiles of the differentially expressed genes (DEGs) related to different biological pathways in the clubroot resistant and susceptible Brassica napus near isogenic lines (NILs) (CR-NILs and CS-NILs) inoculated with Plasmodiophora brassicae pathotype 3H at 7- and 14-days after inoculation (dai). RI=inoculated resistant NILs; SI=inoculated susceptible NILs.

FIG. 12 depicts an agarose gel picture of 92 Brassica napus recombinant-inbred lines (RIL) lines genotyped with simple sequence repeat (SSR) marker SSR-6115 from A09 (30.42 Mb region); Invitrogen 100 bp Ladder (Thermo Fisher) is also included in the gel picture. The band of about 300 bp size, generated by the SSR-6115 marker of the A09 locus, showed co-segregation with clubroot resistance. RP signifies the resistant parent ECD 01 (Brassica rapa var. rapifera); SP signifies the susceptible parent B. napus spring canola line A04-73NA; R and S signifies the resistant and susceptible lines of the RIL population, respectively.

FIG. 13 depicts differentially accumulated proteins (DAPs) in roots of the clubroot resistant (CR) and susceptible (CS) Brassica napus near-isogenic lines (NILs) inoculated with Plasmodiophora brassicae pathotype 3H. RI=inoculated resistant NILs; RC=uninoculated (control) resistant NILs; SI=inoculated susceptible NILs; SC=uninoculated (control) susceptible NILs; dpi=days post inoculation.

### DETAILED DESCRIPTION

Embodiments of the present disclosure are directed to a modified nucleic acid sequence that may confer improved resistance to clubroot disease in canola plants. In some embodiments, the nucleic acid sequence is provided. In other embodiments, a modified canola plant cell comprising the nucleic acid sequence is provided. In yet other embodiments, a modified, canola plant comprising the nucleic acid sequence is provided. As will be appreciated, embodiments of the modified nucleic acid sequence, plant cell, and plant described herein may be used to create, propagate, and cultivate canola plants with enhanced resistance to clubroot disease.

### Definitions

The term “clubroot” is used herein to refer to the disease caused by the pathogen Plasmodiophora brassicae (P. brassicae). As a matter of convenience, the terms “clubroot” and “P. brassicae” may be used interchangeably herein and are intended to be synonymous. Similarly, the terms “clubroot resistance” and “P. brassicae resistance” may be used interchangeably herein and are intended to be synonymous.

As used herein, “clubroot resistance” refers to resistance to one or more Plasmodiophora brassicae isolates, such as, but not limited to, resistance to the Plasmodiophora brassicae pathotypes 3H, 2B, 3A, 3D, 5G, 5X, 5L, 5O, 8E and 8P. In some examples, resistance refers to a reduction in damage caused by P. brassicae infection compared to damage caused on control plants. Damage can be assessed as, for example, formation of club-shaped galls on the roots, occurrence of wilting, stunting, yellowing, premature senescence, etc. In particular, a reduction in damage is manifested in a reduced yield loss when plants are grown under disease pressure in the field, compared to control plants. Such reduction in yield loss can, for example, be due to the fact that the infection, reproduction, spread or survival of the pathogen is reduced or prevented in plants with enhanced resistance. Said resistance may also refer to plants that are completely resistant, i.e., plants on which no disease symptoms are found.

In some examples, for resistance to different pathotypes, disease severity index (DSI, %) values are determined, where DSI <30% is considered as resistant and >70% considered as susceptible.

The term “canola” is used herein to refer to the plant species Brassica napus (B. napus). As such, the terms “canola” and “B. napus” may be used interchangeably herein and are intended to be synonymous.

The term “turnip” is used herein to refer to the plant species Brassica rapa (B. rapa). As such, the terms “turnip” and “B. rapa” may be used interchangeably herein and are intended to be synonymous.

The term “B. napus x B. rapa cross” is used herein to refer to a P. brassicae-resistant B. napus line generated by the hybridization of B. napus A04-73NA and B. rapa cv. Debra/ECD 01, as disclosed in more detail herein.

Embodiments of the modified nucleic acid sequence disclosed herein are derived from chromosomal DNA of the progeny derived from B. napus x B. rapa cross.

The term “nucleic acid sequence” is used herein to refer to any molecule comprising a plurality of nucleotides, including deoxyribonucleic acids (DNA) and/or ribonucleic acids (RNA), regardless of whether such nucleotides are polymerized to form a single strand or double strand, or are naturally-occurring, synthetic, or any combination thereof. Although the present disclosure is directed to a modified nucleic acid sequence derived from chromosomal DNA, it will be appreciated that the nucleic acid sequence may have at least one complementary DNA sequence and at least one analogous RNA sequence. Additionally, the term “nucleic acid sequence” should be understood to refer to any amino acid sequences (e.g. proteins) encoded therein, in addition to any material non-coding regions (e.g. for controlling translation and transcription), which separately or together may comprise one or more genes and/or functional portions.

The term “sequence identity” is used herein to refer to the similarity of two nucleic acid sequences. Two sequences are identical if they have an identical number and order of nucleotides within the sequences. Two sequences may be similar if they have a plurality of nucleotides in an identical order, however, the number of nucleotides may be different (i.e. insertions or deletions) or the identity of some nucleotides at specific positions within the sequence may be different (i.e. substitutions). The degree of similarity between two sequences may be reduced to a numeric value between 0% and 100% sequence identity, with 100% sequence identity denoting two identical sequences. As will be appreciated, the redundant nature of nucleic acids and nucleic acids means that some nucleic acid sequences are materially identical without having a 100% sequence identity. For example, two nucleic acid sequences with 90% sequence identity may encode identical or analogous amino acid sequences and may contain analogous regions for controlling translation and transcription, which result in each nucleic acid sequence having the same biological function without having 100% sequence identity. As such, it will be appreciated that nucleotides may be deleted, added, or substituted within a sequence without materially altering the sequence.

The term “locus” refers to a nucleic acid sequence extending between two nucleic acid positions within a chromosome. In the present disclosure, several loci are provided within B. napus chromosomes, each locus comprising a nucleic acid sequence that confers resistance to clubroot disease in B. napus either alone or in combination with at least one other locus. As a matter of convenience, the modified nucleic acid sequence disclosed herein is defined by its locus or loci within the chromosomes of the B. napus x B. rapa cross rather than a specific sequence listing.

### Modified Nucleic Acid Sequence

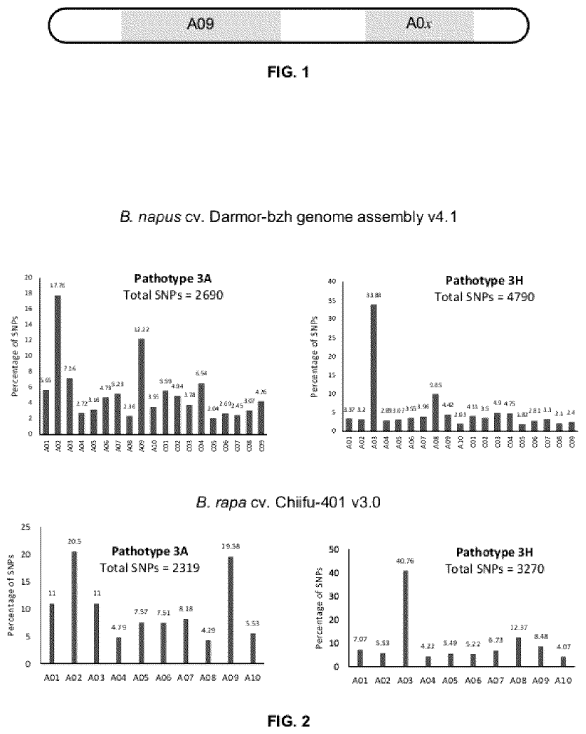

According to embodiments, having regard for FIG. 1, a modified nucleic acid sequence conferring improved resistance to P. brassicae is disclosed, the nucleic acid sequence comprising at least one region having at least 90% sequence identity to the functional portion of B. napus x B. rapa cross chromosomal resistance locus A09. In some embodiments, the nucleic acid sequence may also comprise one or more additional regions, each additional region having at least 90% sequence identity to the functional portion of B. napus x B. rapa cross chromosomal resistance loci A02, A03, or A08. As will be appreciated, insertion of the nucleic acid sequence into B. napus cells and plants will provide plants with enhanced resistance to P. brassicae.

Embodiments of the modified nucleic acid sequence are disclosed herein with reference to loci of the B. napus x B. rapa cross chromosomes produced by the method disclosed in Example 1, below. Although the present disclosure does not include a complete sequence listing of embodiments of the nucleic acid sequence, the method described herein enables the production of the nucleic acid sequence. Further, it will be understood that the loci of the B. napus x B. rapa cross chromosomes are identified herein according to known reference genomes, including genome assemblies of B. napus cv. Darmor-bzh v4.1 and B. rapa cv. Chiifu-401 v3.0. As the reference genomes do not have a 100% sequence identity with the B. napus x B. rapa cross, it will be appreciated that loci of the B. napus x B. rapa cross chromosomes described herein may be approximations and may not describe the exact nucleotide positions of loci within the B. napus x B. rapa cross chromosomes.

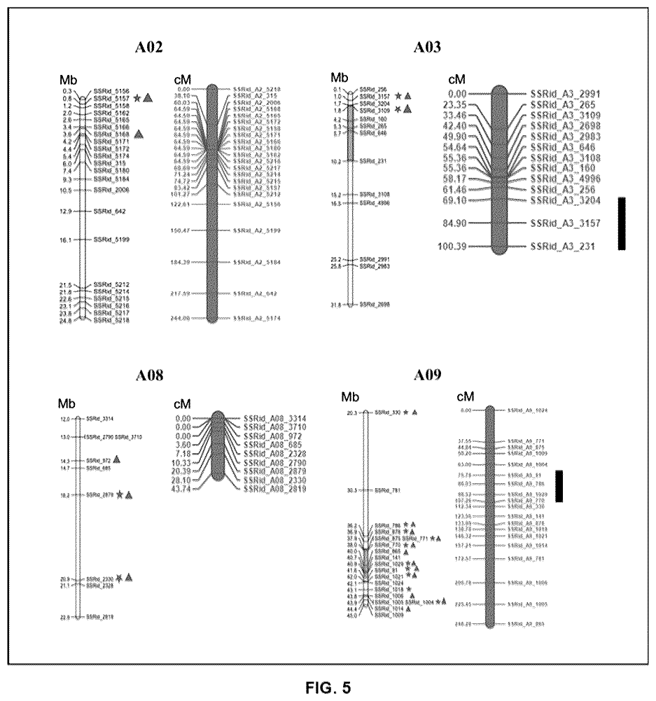

In some embodiments, having regard for FIG. 5, the modified nucleic acid sequence may comprise at least one region derived from B. napus x B. rapa cross chromosome A09, namely the locus extending between nucleotide positions 38.01 Mb to 41.5 Mb within chromosome A09 (the “A09 Clubroot Resistance Locus”). Specifically, the at least one region of the modified nucleic acid sequence may be materially identical to at least one functional portion of the nucleic acid sequence of the A09 Clubroot Resistance Locus such that the modified nucleic acid sequence confers substantially the same clubroot resistance as the A09 Clubroot Resistance Locus (i.e. substantially the same genes, protein coding regions, material non-coding regions, etc., of the A09 Clubroot Resistance Locus). In some embodiments, the at least one region of the modified nucleic acid sequence and the at least one functional portion of the A09 Clubroot Resistance Locus may have a sequence identity of at least 90% (i.e. 90%, 91%, 92%, 93%, 94%, 95%, 96%, 97%, 98%, 99%, or 100%).

In some embodiments, having further regard for FIG. 5, the nucleic acid sequence may also comprise one or more additional regions, each additional region derived from B. napus x B. rapa cross chromosome A02, A03, or A08, as is described in more detail below. In the case of chromosome A02, according to embodiments, the additional region may be derived from the locus extending between nucleotide positions 0.80 Mb to 3.63 Mb within chromosome A02 (the “A02 Clubroot Resistance Locus”). Specifically, the at least one region of the modified nucleic acid sequence may be materially identical to at least one functional portion of the nucleic acid sequence of the A02 Clubroot Resistance Locus such that the modified nucleic acid sequence confers substantially the same clubroot resistance as the at least one functional portion of the A02 Clubroot Resistance Locus (i.e. substantially the same genes, protein coding regions, essential non-coding regions, etc., of the A02 Clubroot Resistance Locus). In some embodiments, the at least one region of the modified nucleic acid sequence and the A02 Clubroot Resistance Locus may have a sequence identity of at least 90% (i.e. 90%, 91%, 92%, 93%, 94%, 95%, 96%, 97%, 98%, 99%, or 100%).

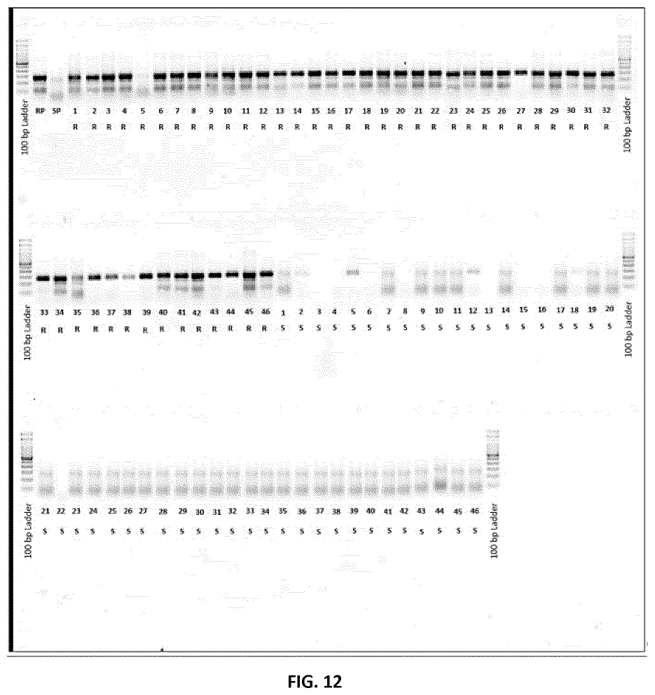

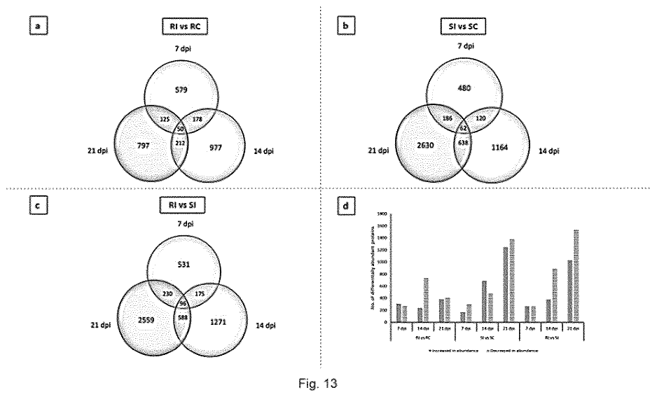

In some examples, SNP-based work identified this A09 locus at 28.7 to 37.3 Mb region (Table 8). In some examples, the RNA-seq work followed by marker work (see FIG. 12) narrowed down this locus to about 30.42 Mb region.

In the case of chromosome A03, according to embodiments, the additional region may be derived from the locus extending between nucleotide positions 1.65 Mb to 10.23 Mb within chromosome A03 (the “A03 Clubroot Resistance Locus”). Specifically, the at least one region of the modified nucleic acid sequence may be materially identical to at least one functional portion of the nucleic acid sequence of the A03 Clubroot Resistance Locus such that the modified nucleic acid sequence confers substantially the same clubroot resistance as the at least one functional portion of the A03 Clubroot Resistance Locus (i.e. substantially the same genes, protein coding regions, essential non-coding regions, etc., of the A03 Clubroot Resistance Locus). In some embodiments, the at least one region of the modified nucleic acid sequence and the A03 Clubroot Resistance Locus may have a sequence identity of at least 90% (i.e. 90%, 91%, 92%, 93%, 94%, 95%, 96%, 97%, 98%, 99%, or 100%).

In some examples, SNP-based work identified this A03 locus at 10.9 to 14.5 Mb region (Table 8).

In the case of chromosome A08, according to embodiments, the additional region may be derived from the locus extending between nucleotide positions 14.34 Mb to 20.87 Mb within chromosome A08 (the “A08 Clubroot Resistance Locus). Specifically, the at least one region of the modified nucleic acid sequence may be materially identical to at least one functional portion of the nucleic acid sequence of the A08 Clubroot Resistance Locus such that the modified nucleic acid sequence confers substantially the same clubroot resistance as the at least one functional portion of the A08 Clubroot Resistance Locus (i.e. substantially the same genes, protein coding regions, essential non-coding regions, etc., of the A08 Clubroot Resistance Locus). In some embodiments, the at least one region of the modified nucleic acid sequence and the A08 Clubroot Resistance Locus may have a sequence identity of at least 90% (i.e. 90%, 91%, 92%, 93%, 94%, 95%, 96%, 97%, 98%, 99%, or 100%).

Although it is contemplated that the nucleic acid sequence may comprise one or more additional regions derived from chromosomes A02, A03, and A08, it should be understood that other clubroot resistance loci are known and the additional regions of the modified nucleic acid disclosed herein may comprise such known loci, in addition to the novel region derived from the at least one functional portion of chromosome A09.

### Modified Canola Plant Cell

According to embodiments, a modified B. napus plant cell conferring resistance to P. brassicae is disclosed, the plant cell comprising at least one nucleic acid sequence having at least 90% sequence identity to B. napus x B. rapa cross chromosomal resistance locus A09 (i.e. one embodiment of the modified nucleic acid sequence disclosed above, as comprised by the modified plant cell). In some embodiments, the plant cell may also comprise one or more additional nucleic acid sequences, each additional nucleic acid sequence having at least 90% sequence identity to B. napus x B. rapa cross chromosomal resistance loci A02, A03, or A08 (i.e. other embodiments of the modified nucleic acid sequence disclosed above, as comprised by the modified plant cell).

In some embodiments, the modified plant cell may comprise the at least one nucleic acid sequence at a substantially identical locus or loci compared to the B. napus x B. rapa cross from which the nucleic acid sequence is derived. In other embodiments, the modified plant cell may comprise the nucleic acid sequence at another locus or loci within its genome (i.e. transgenic locus or loci) or within a non-genomic expression vector.

As will be appreciated, propagating or grafting the plant cell to B. napus plants will provide plants with enhanced resistance to P. brassicae. The modified plant cell may be a product of the method disclosed in Example 1, below.

### Modified Canola Plant

According to embodiments, a modified B. napus plant is disclosed, the plant comprising at least one nucleic acid sequence having at least 90% sequence identity to B. napus x B. rapa cross chromosomal resistance locus A09 (i.e. one embodiment of the modified nucleic acid sequence disclosed above, as comprised by the modified plant). In some embodiments, the plant may comprise one or more additional nucleic acid sequences, each additional nucleic acid sequence having at least 90% sequence identity to B. napus x B. rapa cross chromosomal resistance loci A02, A03, or A08 (i.e. other embodiments of the modified nucleic acid sequence disclosed above, as comprised by the modified plant).

In some embodiments, the modified plant may comprise the at least one nucleic acid sequence at a substantially identical locus or loci compared to the B. napus x B. rapa cross from which the nucleic acid sequence is derived. In other embodiments, the modified plant may comprise the nucleic acid sequence at another locus or loci within its genome (i.e. transgenic locus or loci) or within a non-genomic expression vector.

As will be appreciated, the plant has enhanced resistance to P. brassicae. The modified plant may be the product of the method disclosed in Example 1, below.

### Example 1—Embodiments of a Method for Producing a Modified Nucleic Acid Sequence, Plant Cell, and Plant

According to embodiments, a method of producing a modified nucleic acid sequence conferring resistance to P. brassicae is disclosed. In some embodiments, the method comprises producing a modified canola plant cell or modified canola plant that comprises the modified nucleic acid sequence.

As will be appreciated, the methods disclosed herein are directed to a preferred embodiment and other embodiments, as appropriate. Some steps of the methods disclosed herein may be substituted or modified and the present disclosure is not intended to restrict or limit the use of any such known substitutions or modifications, provided that such methods produce the modified nucleic acid sequence, modified plant cell, or modified plant disclosed herein. Conversely, some steps of the methods disclosed herein are essential, as will be appreciated.

### Plant Material and Population Development

In some embodiments, the modified nucleic acid sequence may be produced by cross breeding or hybridizing two parent lines of B. napus and B. rapa. In such embodiments, the parent lines may be a clubroot susceptible spring B. napus (AACC, 2n=38) canola-quality (zero erucic acid in oil and low glucosinolate (GSL) in seed meal)) line A04-73NA and a clubroot-resistant B. rapa var. rapifera turnip accession of the European Clubroot Differential (ECD) set (accession ECD 01 or cv. Debra). ECD 01 is known to be completely resistant to multiple pathotypes including pathotype 3H and 3A (for example, disease score=0, disease severity index or DSI=0%). The resulting B. napus x B. rapa interspecific cross may be made by using A04-73NA as female, and obtaining F1 seeds. High meiotic anomalies are expected in allotriploid (AAC) F1 plants; therefore, the F1 plants may be backcrossed to A04-73NA to generate BC1 plants and to achieve canola-quality clubroot resistant euploid (2n=38) B. napus lines. These lines may also be obtained in self-pollinated progeny of the F1 plants, i.e., without backcrossing the F1 plants. However, loss of about 55% of the B. rapa alleles can occur in early generation progeny of the F1 plants (Attri and Rahman 2016), which is apparently due to high aneuploidy in the F1-derived population. Therefore, backcrossing the F1 to the B. napus parent is preferred to minimize the aneuploidy and loss of the B. rapa alleles, and thus, to achieve euploid B. napus plants from this interspecific cross with minimum loss of the B. rapa alleles. As will be appreciated, however, the loss of B. rapa alleles may also be mitigated to some extent by growing a large early-generation population. The BC1 plants developed by embodiments of the methods disclosed herein may be self-pollinated for multiple generations with selection for spring growth habit, canola-quality traits, and resistance to P. brassicae pathotype 3H in the early generations, and resistance to 3A and other pathotypes in later generations. The details of development of this population are known (Kaur et al. 2022). In some embodiments, a total of 632 BC1F6 recombinant inbred lines (RILs) resistant to pathotype 3H and 671 BC1F7 RILs resistant to pathotype 3A may be developed from the presently disclosed interspecific cross. Advanced generation RILs susceptible to these two pathotypes (100 lines susceptible to pathotype 3H and 60 lines susceptible to pathotype 3A) may also developed from the presently disclosed interspecific cross for mapping of the resistance by using the whole genome resequencing (WGRS) followed by quantitative trait loci (QTL) mapping approaches.

### Whole Genome Resequencing (WGRS) and QTL Mapping

In some embodiments, based on resistance phenotype of the RILs, the following bulks of DNA samples may be prepared for WGRS:
(i) the resistant parent B. rapa var. rapifera ECD01 (cv. Debra) (RP) (resistant to pathotypes 3H and 3A),
(ii) the susceptible parent B. napus canola line A03-73NA (SP) (susceptible to both pathotypes),
(iii) a bulk DNA of 50 RILs resistant to pathotype 3H (RB-3H),
(iv) a bulk DNA of 50 RILs susceptible to pathotype 3H (SB-3H),
(v) a bulk DNA of 50 RILs resistant to pathotype 3A (RB-3A), and
(vi) a bulk DNA of 50 RILs susceptible to pathotype 3A (SB-3A).

For extraction of DNA, according to embodiments, the plant materials may be grown in a greenhouse, and young leaves of 3-4-week-old plants may be used; DNA may be extracted using a known, modified SDS DNA extraction protocol (Kotchoni and Gachomo 2009). The quality and quantity of DNA may be determined with a ND-2000 Nanodrop spectrophotometer (Nanodrop Technologies Inc., Wilmington, Del., USA) and Qubit 3.0 fluorometer (ThermoFisher Scientific Inc., USA).

For sequencing, according to embodiments, DNA libraries may be prepared using TruSeq DNA Sample Prep Kit and sequencing may be carried out using Illumina HiSeq 4000 platform to generate 150 bp paired-end reads. Library preparation, sequencing and data analysis, including alignment (mapping) of the sequence reads to the reference genome and single nucleotide polymorphism (SNP) calling may be carried out by Genewiz (Genewiz; www.qenewiz.com). In some embodiments, the resulting six samples may be sequenced 30× to increase the sequencing depth and breadth, and thus, to obtain more accurate results.

In some embodiments, the adapter sequences and nucleotides with poor quality may be trimmed off using Trimmomatic v0.36 (Bolger et al. 2014) and, in a preferred embodiment, only high-quality sequencing reads are used for downstream analysis. The reads shorter than 35 bp may also be discarded. The trimmed reads of the sample RP may be mapped to two reference genomes, the winter B. napus cultivar Darmor-bzh whole-genome assembly v4.1 (Chalhoub et al. 2018) and the B. rapa cv. Chiffu-401 whole-genome assembly v3.0 (Wang et al. 2011; Zhang et al. 2018), using Burrows-Wheeler Aligner (BWA) v0.7.12 (Li and Durbin 2009). The files obtained from BWA alignment may be processed and the mapped reads at each nucleotide position may be extracted using mpileup function in Samtools v1.3.1 (Li et al. 2009). The variants between the RP reads and the A genome of the reference genome assemblies may be called using VarScan v2.3.9 (Koboldt et al. 2012) with the following settings: minimum coverage 10, minimum number of reads 7, minimum variant frequency 10%, and the highest p-value 0.05. Based on this, two sequence assemblies of the RP, viz. RPA-Chiffu based on the reference genome B. rapa cv. Chiffu-401 and RPA-Darmor based on the reference genome B. napus cv. Darmor-bzh, may be developed. The trimmed reads of the bulks, RB-3H, SB-3H, RB-3A and SB-3A may be individually aligned to the two RPA's and SNPs called for these bulks, and SNP index may be calculated.

In some embodiments, the SNP index may be the ratio of SNP variants at each SNP position. This ratio may be calculated for each of the four bulks RB-3H, SB-3H, RB-3A and SB-3A at each SNP position by comparing with the RPA assemblies using the following formula (Abe et al. 2012):

$$SNP index = \frac{Counts of alternate ( alternate to RPA ) nucleotide read in bulk}{Total counts of nucleotide reads in bulk}$$

This calculation may be carried out separately using the SNP data obtained based on the two reference genomes disclosed above.

In some embodiments, by using SNP index data of the RB-3H and SB-3H (for resistance to pathotype 3H) and RB-3A and SB-3A (for resistance to pathotype 3A), the ΔSNP index may be calculated by subtracting SNP index of the resistant bulk from the SNP index of the susceptible bulk, and this may be done for these two groups (RB-3H vs. SB-3H and RB-3A vs SB-3A) separately. The SNP positions with ΔSNP index=−1 may be considered the SNPs associated with the trait (clubroot resistance) of interest. The ΔSNP index of −1 may denote that the allele in the resistant bulk is the same as that of the resistant parent while a different allele is present in the susceptible bulk.

### Designing of Primers and Polymerase Chain Reaction (PCR)

In some embodiments, a total of 391 simple sequence repeat (SSR) markers and 64 SNP allele-specific (SNP-AS) markers may be used to genotype the pathotype 3A resistant and susceptible RIL population that may be used for WGRS. SNP-AS primers and SSR primers may be designed based on ΔSNP index of the resistant and susceptible bulks using the software program BatchPrimer3 (You et al. 2008); other SSR primers may be obtained from other sources (e.g. the University of Alberta Canola Program, Edmonton, Alberta, Canada) (e.g. Hobson and Rahman 2016). The SSR primers may be designed from the genomic regions associated with resistance as well as carrying the TIR-NBS-LRR (Toll/Interleukin1-Nucleotide Binding Site-Leucine Rich Repeat) genes. While designing the SSR primers, the universal M13 sequence 5′-CACGACGTTGTAAAACGAC-3′ (SEQ ID NO: 1) fluorescently labelled with infrared FAM, VIC, NED and PET (IRD; MWG-Biotech AG, Edensburg, Germany) may be added to the forward primer.

In some embodiments, polymerase chain reaction (PCR) may be carried out in a total volume of 14 μl, where each reaction mixture includes 50 ng (25 ng/μl×2 μl) of DNA, 2.5 μl of 1×PCR buffer, 0.125 μl of 20 mM dNTPs, 1.25 μl of 50 mM MgCl2, 0.5 μl each of forward and reverse primer, 0.25 pmol/L of labelling dye (FAM, VIC, NED and PET; Applied Biosystem, Foster City, California), 0.125 U of GoTaq DNA polymerase and 6.75 μl of nuclease free water. The amplified products may be detected using a capillary ABI Sequencer No. 3730 (Applied Biosystems) and may be scored and analysed using GeneMapper software v5.0. On the other hand, the amplified products of the SNP-AS primers may be separated on 2.5-3.0% agarose gel, stained with SYBR Green1, and imaged using Typhoon FLA9500 laser scanner (GE Healthcare).

### Identification of Polymorphic Markers, Genotyping, and Mapping the Clubroot Resistance Loci

In some embodiments, the above-mentioned SSR and SNP-AS markers may be tested on the parents ECD 01 and A04-73NA to identify the polymorphic markers, and the polymorphic markers may be used to genotype the RIL mapping population, which may include 47 lines carrying resistance to pathotype 3A (most of them may also be resistant to pathotype 3H) and 47 susceptible lines. By using the genotypic data, a genetic linkage map may be constructed using the software program QTL Ici Mapping v4.2 (Meng et al. 2015). Segregation distorted markers may be removed while constructing the frame-work map; however, they may be individually added later on the maps based on their linkage group. A threshold value of logarithm of the odds (LOD) 4.0 and rippling with a window size of 5 cM may be used to obtain the linkage map; in this, Kosambi mapping function (Kosambi, 2016) may be used to estimate recombination frequency.

In some embodiments, to identify the genomic region associated with clubroot resistance, the genotypic (marker) and phenotypic (clubroot resistance) data may be analysed using the same QTL Ici Mapping v4.2 software program (Meng et al. 2015) with walking speed of 0.4 cM and stepwise regression probability of 0.004; the threshold for LOD (p-value <0.05) may be obtained from 1,000 permutations to declare the location of the QTL (Churchill and Deorge 1994). The QTL may be detected following the ICIM-ADD, ICIM-EPI and SMA method of QTL Ici Mapping v4.2. The likelihood of a QTL and its effects at every 0.1 cM intervals may be estimated in a window of 10 cM. The percentage phenotypic variation explained by additive effect of the QTL may be obtained from an inclusive composite interval mapping model (Li et. al. 2008).

In addition to QTL mapping approach, according to embodiments, single marker analysis (SMA) and genome-wide association study (GWAS) may be carried out using the above-mentioned SSR and SNP-AS markers to identify the genomic regions conferring resistance to clubroot disease. SMA may be carried out using the marker and phenotypic data, and the analysis may be performed using the software program “R”. Genome-wide association study (GWAS) may be carried out using the software program TASSELv5.2.73 (Bradbury et al. 2007). To detect the association of the naïve, Q, K and PCA model may be performed using the general linear model (GLM) method, and the K+Q and K+PCA models may be performed with a mixed linear model (MLM) method. The additive effect of the QTL may be obtained from the GLM output table provided by Tassel v.4 using the additive genetic variance and kinship matrix. The p-value threshold obtained from the GLM table may be converted to LOD score. Subsequently, a genome-wide significance threshold level greater than p-value=1.0×10⁻⁴□□ LOD of 4.0 may be set to detect the markers significantly associated with resistance. The phenotypic variance explained by the significant QTL markers may be calculated using the R² values.

### Construction of a Physical Map

In some embodiments, the forward and reverse sequences may be aligned to the B. rapa Chiffu-401 whole genome assembly v3.0 using BLASTn tool in Brassica database (BRAD; https://brassicadb.cn) to determine the physical position of the markers. Following the alignment, the distance (length) between the forward and the reverse primer may be deduced and if it was similar to the known amplicon size of the marker, the position of the marker may be considered its physical position in the genome. By using this information, physical maps of the chromosomes may be constructed.

### Marker Development and Genotyping

Based on results from WGRS-based QTL mapping, putative disease resistance genes from about 1 to 10 Mb region of A03 and about 37 to 40 Mb region of A09 were identified, and sequences of 244 genes were downloaded from BRAD (http://brassicadb.cn/#/ResistanceGene/) and used to find their homologues in B. napus by using the ‘Blat’ function in Genoscope Brassica napus Genome Browser (https://www.genoscope.cns.fr/blat-server/cgi-bin/colza/webBlat). Based on this, a total of 38 genes were identified from the two QTL regions. Search for functional domain was carried out in these genes using SMART (Simple Modular Architecture Research Tool) (http://smart.embl-heidelberg.de/) and the domains were detected in 29 of the genes (18 from A03 and 11 from A09); these 29 genes were retained and the other discarded. In addition to this, gene annotation analysis was performed using sequences of the genes from the A09 QTL region using Blast2GO (Conesa et al. 2005) and 14 genes related to defense response were identified. Thus, a total of 43 (29+14) genes were identified from the two genomic regions and were used to design molecular markers.

A total of 107 SSR primers pairs (40 from A03 and 67 from A09) were designed based on the above-mentioned 43 genes using the software program Primer3Plus (Untergasser et al. 2012). In addition to this, 26 SNP allele-specific (AS-SNP) primers were designed from the A09 QTL region. The RIL mapping population was genotyped with these markers for linkage association with resistance.

### SNP Genotyping and QTL Mapping

The RILs which have been used for WGRS were genotyped by 19 K SNP (single nucleotide polymorphism) array for mapping of the resistance through genome-wide association study (GWAS). The genotyping panel included 46 RILs resistant to pathotype 3A and 46 susceptible RILs, as well as the clubroot resistant turnip parent ECD 01 (cv. Debra) and the clubroot susceptible canola parent A04-73NA.

Leaf sample of each of the above-mentioned accessions were collected in a 96-well plate, and the materials were dried in an oven at 60° C. for about 4-5 hours. The samples were shipped to SGS North America Inc, Brookings, USA (https://www.sgs.com/en-us) for DNA extraction and sequencing using Illumina HiSeq 2500 platform. The sequence reads of the samples were mapped to the B. napus pangenome (Hurgobin et al. 2018) and SNP analysis was carried out using Brassica 19 K array. The heterozygous SNP loci and calls with minor allele frequency (MAF) of <5% were considered as missing data and were eliminated from further analysis. Based on this, a total of 18,566 SNPs were identified. However, the SNPs monomorphic between the parents ECD 01 (cv. Debra) and A04-73NA were discarded and this left 4144 SNPs, which were used for genome-wide association mapping of the resistance.

Principal component (PCA) and kinship analysis of the 92 RILs was carried out using the 4144 SNP markers, and the analysis was carried out using Genome Association and Prediction Integrated Tool (GAPIT) package (Lipka et al. 2012) in R v3.6.0. Association mapping of the resistance was performed using the method Bayesian-information and Linkage-disequilibrium Iteratively Nested Keyway (BLINK) in R v3.6.0 (Huang et al. 2019) taking population structure (PCs) and kinship coefficients (genetic relatedness) into account. Compared to other association mapping methods, such as FarmCPU, BLINK improves the statistical power of genome-wide association study as well as reduces the computing time (Huang et al. 2019); therefore, this method was used in this study. The Bonferroni-corrected threshold of p-value was adjusted to 0.000001 (−log 10 (p)=6.0) to test the significance of association between the markers and the resistance. The parameter of permutations=2000 was set to break the relationship with the genotype, and this also controlled the experiment-wise type I errors (false positives, i.e. found significant association when there is no association). The p (−log 10P) values of all SNP markers were used to draw the Manhattan plot. Quantile-quantile (Q-Q) of the observed and expected p-values were plotted to assess the adequacy of the fitted normal straight line to the markers. In this, any deviation from the expected line denotes the SNPs to be associated with clubroot resistance.

### Marker Development and Genotyping

Based on results from the SNP-based QTL mapping, putative disease resistance genes from about 10 to 15 Mb region of A03 and about 27 to 37 Mb region of A09 were identified, and molecular makers were designed following the approach described above for genotyping the RIL mapping population for linkage association of the markers with resistance.

### Epistasis Analysis of the Clubroot Resistance Loci

Based on QTL mapping of the resistance, digenic epistasis analysis for additive×additive interaction effect was carried out using QTL IciMapping v4.2 (Meng et al. 2015) with a walking speed of 1.0 cM and stepwise regression probability of 0.004; the threshold for logarithm of the odds (LOD) (p value <0.05) was obtained from 1,000 permutations.

### Development of Near-Isogenic Lines (NILs)

One of the advanced generation canola lines derived from the (B. napus A04-73NA×B. rapa turnip)×B. napus interspecific cross carrying resistance to P. brassicae pathotypes 3A and 3H was crossed to the B. napus parent A04-73NA, and the F₁ was recurrently backcrossed to A04-73NA for four-times and the resulting BC₄ plants were self-pollinated for two generations and BC₄F₃ near-isogenic lines (NILs) were developed. For this, in each backcross generation, plants exhibiting resistance to pathotype 3H were backcrossed to the recurrent parent A04-73NA. The resulting BC₄F₃ NILs showing resistance or susceptibility to pathotype 3H were evaluated for resistance to 10 P. brassicae pathotypes, and were also used for transcriptome (RNA-Seq) and proteome analyses. The pathotype 3H-resistant NILs also carried resistance to pathotype 3A.

### Transcriptome Analysis of the NILs and Molecular Marker Development

Three homozygous clubroot resistant (CR-NILs) and three homozygous clubroot susceptible (CS-NILs) BC₄F₃ NILs were used for transcriptome (RNA-seq) analysis for identification of the differentially expressed genes (DEGs) and to develop molecular markers based on the DEGs; these two types of NILs hereafter referred to as CR-NILs and CS-NILs, respectively. The NILs were grown in a greenhouse at the University of Alberta and were inoculated with P. brassicae pathotype 3H at seven days after germination; the control plants were left un-inoculated. Our previous research has shown that primary infection on secondary roots began at 7 days after inoculation (dai) and secondary plasmodia could be found at 14 dai (Summanwar et al. 2020). Based on this, the root samples collected at these two time points were used for RNA-seq analysis, and this included a total of 24 samples: [2 genotypes×2 time points (7- and 14-dai)×3 biological replicates×2 treatments (control and P. brassicae-inoculated)]. Each root sample included a pool of three NILs (either resistant or susceptible) and seven plants of each NIL. Thus, the total number of plants used for this experiment was 2×2×3×2×3×7=504 plants.

The RNA of the 24 root samples was extracted using Trizol reagent (Invitrogen, MA, USA) following the manufacturer's protocol. Library preparation, first and second strand cDNA synthesis, and 2×150-bp paired-end read sequencing of the samples using Illumina HiSeq 2500 platform was carried out by Novogene, U.S.A. (https://en.novogene.com/). The sequence reads were mapped to the B. napus cv. Darmor-bzh v4.1 reference genome (Chalhoub et al. 2014; Kim et al. 2015; Pertea et al. 2015). Quantification of the level of gene expression for each sample was carried out based on Fragments per Kilobase of transcript sequence per Million base pairs sequenced (FPKM). The differentially expressed genes (DEGs) between the inoculated (I) and control (C) samples of the CR-(R) and CS-NILs (S) at 7- and 14-dai were detected from the following comparisons: RI vs. RC, SI vs. SC, and RI vs. SI. The Gene Ontology (GO) and Kyoto Encyclopedia of Genes and Genomes (KEGG) enrichment analyses were carried out (Yu et al. 2012; Kanehisa and Goto, 2000) to identify the biosynthetic pathways and the key genes to be involved in disease resistance.

### Marker Development and Genotyping

The DEGs and their genomic locations were used to design molecular markers to investigate the linkage association of the markers/DEGs with clubroot resistance. For this, SSR markers were designed based on the DEGs from the A03 and A09 QTL regions using the software program BatchPrimer3 (You et al. 2008). The markers were tested for polymorphism between the resistant parent ECD 01 (Debra) and the susceptible parent A04-73NA, and the polymorphic markers were used to genotype the resistant and susceptible lines of the RIL population for linkage association of the markers with resistance.

### Proteome Analysis of the NILs

The three homozygous clubroot resistant (CR-NILs) and three susceptible (CS-NILs) BC₄F₃ NILs, which has been used for transcriptome (RNA-seq) analysis, were used for comparative proteomics analysis to identify the proteins playing a role in clubroot resistance. For this, the NILs were inoculated with P. brassicae pathotype 3H; the control plants were treated with sterile water. Roots of the inoculated (I) and control (C) CR- and CS-NILs were collected at 7-, 14- and 21 days post inoculation (dpi) and used for this study. According to Summanwar et al. (2019), primary infection occurs after 6 dpi, secondary plasmodia become visible at 14 dpi, and resting spores become visible at about 21 dpi; therefore, we have chosen these three time points. Thus, the experimental materials included a total of 48 root samples [2 genotypes×3 time points (7-, 14- and 21 dpi)×4 biological replicates×2 treatments (control and P. brassicae-inoculated)]. Each root sample included a bulk of three NILs and seven plants of each NIL. The protein of the samples was isolated, digested with trypsin and subjected to liquid chromatography with tandem mass spectrometry analysis; the details of this methodology can be found in Uhrig et al. (2019). Acquisition of the liquid chromatography-mass spectrometry proteomics data was done following Mehta et al. (2022). The published B. napus Westar proteome (Song et al. 2020) was used as a reference to identify the differentially abundant proteins. Functional annotation of the proteins was carried out using Kyoto Encyclopedia of Genes and Genomes (KEGG; https://www.genome.jp/kegg/), Pfam (Mistry et al. 2020) and SwissProt (https://www. https://www.expasy.org/resources/uniprotkb-swiss-prot) databases.

### Results

### Whole Genome Resequencing (WGRS) and QTL Mapping

In some embodiments, the WGRS may generate about 81.8 to 192.8 million reads of which 94.6 to 99.3% of the reads may be mapped to the reference genomes (Table 1).

TABLE 1
Summary of whole-genome resequencing (WGRS) of the
parental lines and the bulks for resistance to Plasmodiophora brassicae
pathotype 3H and 3A.

| | | | |
|---|---|---|---|
| Samples and | | | |
| reference | Total no. | No. mapped | % mapped |
| genome | reads | reads | reads |

B. napus cv. Darmor-bzh v.4.1

| | | | |
|---|---|---|---|
| RP | 145,873,733 | 142,487,403 | 97.7 |
| SP | 81,884,834 | 80,989,458 | 98.9 |
| RB-3H | 155,152,936 | 153,746,348 | 99.1 |
| RB-3A | 122,863,908 | 121,791,102 | 99.1 |
| SB-3H | 190,548,537 | 189,123,962 | 99.3 |
| SB-3A | 130,354,454 | 129,119,261 | 99.1 |

B. rapa cv. Chiifu-401 v3.0

| | | | |
|---|---|---|---|
| RP | 144,745,045 | 140,035,425 | 96.7 |
| SP | 82,379,743 | 78,273,444 | 95.0 |
| RB-3H | 156,999,003 | 148,653,472 | 94.7 |
| RB-3A | 123,854,338 | 117,782,124 | 95.1 |
| SB-3H | 192,869,610 | 183,130,403 | 95.0 |
| SB-3A | 131,576,834 | 124,903,038 | 94.9 |

In some embodiments, to identify the candidate genomic regions controlling resistance to P. brassicae pathotype 3H and 3A, SNP index for each SNP position may be calculated for each bulk by comparing them with the RPK assembly following known method (Pandey et al. (2017)). The total number of polymorphic SNPs that may be detected in the bulks differing for resistance to the two pathotypes (3H and 3A) by using the two reference genomes (Chiifu-401 v3.0 and Darmor-bzh v.4.1) are presented in Table 2. While using the B. rapa cv. Chiifu-401 reference genome, about 80% greater number of polymorphic SNPs may be detected as compared to the number of SNPs detected using the B. napus cv. Darmor reference genome.

TABLE 2
Total number of polymorphic SNPs detected by using
B. napus cv. Darmor-bzh v.4.1 and B. rapa
cv. Chiifu-401 v3.0 genome assemblies.

| | B. napus cv. | B. rapa cv. |
|---|---|---|
| | Darmor-bzh v.4.1 | Chifu-401 v3.0 |
| Pathotype 3A | 459,777 | 821,784 |
| resistance | | |
| Pathotype 3H | 775,262 | 1,416,058 |
| resistance | | |

In some embodiments, ΔSNP index for each of the polymorphic SNPs (Table 2) may be calculated. From this, about 4,000 SNPs may be identified to be associated with resistance to pathotype 3H and about 2,500 SNPs to be associated with resistance to 3A (Table 3) based on read depth of ≥10 and ΔSNP index=−1.

TABLE 3
Total number of SNPs with ΔSNP index = −1
detected using B. napus cv. Darmor-bzh v.4.1
and B. rapa cv. Chiifu-401 v3.0 genome assemblies.

| | B. napus cv. | B. rapa cv. |
|---|---|---|
| | Darmor-bzh v.4.1 | Chiifu-401 v3.0 |
| Pathotype 3H | 4,793 | 3,270 |
| Pathotype 3A | 2,690 | 2,319 |

The distribution of polymorphic SNPs with ΔSNP index=−1 (Table 3) across the 19 B. napus chromosomes is presented in FIG. 2, according to embodiments. For resistance to pathotype 3H, the greatest number of SNPs may be detected from chromosome A03 (33.88 or 40.76%) followed by A08 (9.85 or 12.37%). For resistance to pathotype 3A, the greatest number of SNPs may be detected from chromosomes A02 (17.76 or 20.5%) followed by A09 (12.22 or 19.58%); however, a considerable number of SNPs may also be detected from A03 (7.16 or 11.0%) (FIG. 2). As will be appreciated, the four A genome chromosomes carry resistance to these two pathotypes. In some embodiments, SNPs in the C genome chromosomes using the Darmor-bzh v.4.1 genome assembly may also be detected; however, their occurrence in most cases may be low (less than 5% of the total). Also, the clubroot resistance in the resistant lines may be introgressed from the A genome of B. rapa; therefore, the C-genome SNPs may be ignored.

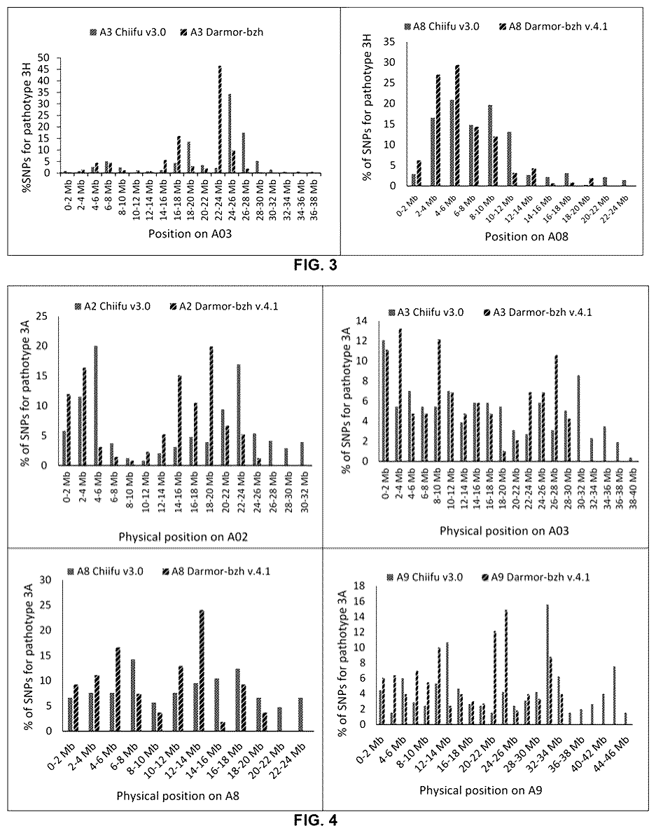

The distribution of the SNPs (based on ΔSNP index), to be associated with resistance to pathotype 3H, along the chromosome A03 and A08 is presented in FIG. 3, according to embodiments. The SNPs of A03 may be largely concentrated in the genomic region of about 16-30 Mb position, and the SNPs of A08 may be largely concentrated on 2-14 Mb region.

In case of resistance to pathotype 3A, the distribution of the SNPs based on ΔSNP index of the chromosomes A02, A03, A08 and A09 is presented in FIG. 4, according to embodiments. The SNPs of A02 may be concentrated in the genomic region of about 0-6 and 14-24 Mb regions, the A03 SNPs may be mostly distributed in a wide region with peaks at about 0-4, 8-10 and 24-32 Mb regions, the A08 SNPs may be distributed at 2-8 and 10-20 Mb regions, and the A09 SNPs may be distributed in a wide region with peaks at about 10-14, 20-24, 30-34 and 36-46 Mb regions indicating that these genomic regions might be associated with resistance to pathotype 3A. Mapping of these chromosome regions may be carried out by genotyping the mapping population with molecular markers from these chromosomes.

Mapping of the Loci Conferring Resistance to P. brassicae Pathotype 3A

For mapping of the clubroot resistance loci, according to embodiments, a total of 391 SSR and SNP allele-specific (SNP-AS) markers from chromosome A02, A03, A08 and A09 may be tested for polymorphism between parents (ECD-1 and A04-73NA) where 70.2% markers may be found to be polymorphic (Table 4). Percent polymorphic marker may be calculated using the following formula: % polymorphic marker=[(No. polymorphic marker)/(Total no. marker tested−No. of marker failed to amplify in both parents)]×100.

TABLE 4
Summary of parental polymorphism using SSR and SNP
allele-specific primers on four chromosomes for resistance
to Plasmodiophora brassicae pathotype 3A.

| | | No. of | No. of | Non- | % of |
|---|---|---|---|---|---|
| Chromo- | Marker | markers | polymorphic | amplified | markers |
| some | type | tested | markers | markers | polymorphic |
| A02 | SSR | 102 | 50 | 18 | 59.5 |
| A02 | SNP-AS | 32 | 14 | 3 | 48.3 |
| A03 | SSR | 138 | 115 | 15 | 93.5 |
| A08 | SSR | 13 | 9 | 4 | 100.0 |
| A09 | SSR | 106 | 50 | 12 | 53.2 |
| Total | | 391 | 238 | 52 | 70.20 |

In some embodiments, based on parental polymorphism, 45 markers from A02, 14 from A03, 9 from A08, and 45 from A09 chromosomes may be used to genotype the mapping population of 47 resistant and 47 susceptible lines, and the genotypic data may be used for construction of genetic linkage map of the four chromosomes (FIG. 5). The physical map of all four chromosomes may be constructed based on the physical positions of the markers from the linkage map (FIG. 5). The A02 linkage map may include 22 markers, A03 may include 13 markers, A08 may include 9 markers, and A09 may include 19 markers.

In some embodiments, by using the genotypic and phenotypic (resistance to pathotype 3A) data, QTL analysis may be performed using QTL IciMapping software. Based on this, one locus on A03 and one on A09 may be identified to be associated with resistance to P. brassicae pathotype 3A (Table 5). These two loci may be identified with LOD score of greater than 6.0. The A03 and A09 loci may explain about 10% and 33% of the total phenotypic variance and exert additive effect of −16.9 and −27.3% DSI, respectively; the minus value may indicate that the B. rapa allele of each of the locus reduced 16.9% and 27.3% DSI. Based on contribution of these loci to the total phenotypic variance and their additive effect to resistance to pathotype 3A, these two loci may be considered as major loci; however, the effect of the A09 locus may be 61.5% greater than the effect of the A03 locus. In addition to these two loci, the genomic regions of A02 and A08 affecting resistance to pathotype 3A may be identified through SMA and GWAS (Table 6, 7).

In some embodiments, the physical positions of these loci may be determined based on reference genome of B. rapa cv. Chiffu v3.0. The A03 and A09 loci identified through QTL mapping approach could be positioned at 1.65-10.23 Mb and at 38.01-41.5 Mb, respectively (Table 5). The physical position of the markers identified through SMA and GWAS may further confirm the position of these two loci (Table 6, 7). Among these markers, the SSRid_A9_330 of A09 identified through GWAS may be positioned at 20.25 Mb; however, the position of this marker on the linkage map may be close to the marker SSRid_A9_770 positioned at 38.01 Mb (FIG. 5). Therefore, SSRid_A9_330 may be located close to the genomic region of SSRid_A9_770. The physical position of the A02 QTL identified through SMA and GWAS approaches may be 0.80 Mb and 0.81-3.63 Mb, respectively; and the position of the A08 QTL identified through these two approaches may be 16.21-20.87 Mb and 14.34-20.87 Mb, respectively.

TABLE 5
Quantitative trait loci (QTL) for resistance to Plasmodiophora brassicae pathotype
3A detected using a Brassica napus recombinant inbred line (RIL) population (n = 94)
derived from (B. napus × B. rapa turnip) × B. napus interspecific
cross; analysis carried out using inclusive composite interval mapping (ICIM-Add) method.

| | | | | | | | Physical position | |
|---|---|---|---|---|---|---|---|---|
| | Position | | | | PVE | | on | |
| Chromosome | cM | Left marker | Right marker | LOD | (%) | Add | B. rapa Chiffu v3.0 | |
| A03 | 84.9 | SSRid_A3_3204 | SSRid_A3_231 | 6.1 | 10.4 | −16.9 | 1.65-10.23 | Mb |
| A09 | 98.5 | SSRid_A09_91 | SSRid_A09_770 | 18.5 | 33.6 | −27.3 | 38.01-41.5 | Mb |

Note.
LOD = logarithm of the odd; PVE = phenotypic variation explained by the QTL.

Add=Estimated additive effect of the QTL; negative sign indicates ‘B. rapa turnip’ allele reduces disease severity index (DSI), i.e., contribute to resistance.

TABLE 6
Quantitative trait loci (QTL) detected for resistance to
Plasmodiophora brassicae pathotype 3A by using a Brassica napus
recombinant inbred line (RIL) population (n = 94) derived
from (B. napus × B. rapa turnip) × B. napus interspecific
cross and following single marker analysis approach.

| | | | | | Physical |
|---|---|---|---|---|---|
| | | | | | position |
| Chromo- | | | R² | | (Mb) on B. rapa |
| some | Marker name | LOD | (%) | Add | Chiffu v3.0 |
| A02 | SSRid_A2_5157 | 4.3 | 18.8 | −22.3 | 0.81 |
| A03 | SSRid_A3_3109 | 3.4 | 15.3 | −24.7 | 1.78 |
| A03 | SSRid_A3_3157 | 6.6 | 27.5 | −27.8 | 0.98 |
| A08 | SSRid_A08_2879 | 4.8 | 21.0 | −25.6 | 16.21 |
| A08 | SSRid_A08_2330 | 5.9 | 25.0 | −29.5 | 20.87 |
| A09 | SSRid_A9_771 | 6.0 | 25.2 | −25.6 | 37.93 |
| A09 | SSRid_A9_91 | 11.5 | 42.5 | −32.0 | 41.55 |
| A09 | SSRid_A9_786 | 22.0 | 65.1 | −38.4 | 36.23 |
| A09 | SSRid_A9_1029 | 8.8 | 35.0 | −28.1 | 40.94 |
| A09 | SSRid_A9_770 | 7.0 | 29.0 | −26.0 | 38.01 |
| A09 | SSRid_A9_330 | 5.2 | 22.3 | −22.8 | 20.25 |
| A09 | SSRid_A9_878 | 6.7 | 28.0 | −30.0 | 36.80 |
| A09 | SSRid_A9_1018 | 5.3 | 23.0 | −27.2 | 43.12 |
| A09 | SSRid_A9_1021 | 7.7 | 31.0 | −28.3 | 42.00 |
| A09 | SSRid_A9_1005 | 5.0 | 21.0 | −21.7 | 43.86 |

Note.
LOD = logarithm of the odd; R² = phenotypic variation explained by the QTL.

Add=Estimated additive effect of the QTL; negative sign indicates ‘B. rapa turnip’ allele reduces disease severity index (DSI), i.e., contribute to resistance.

TABLE 7
Quantitative trait loci (QTL) detected for resistance
to Plasmodiophora brassicae pathotype 3A by using
a Brassica napus recombinant inbred line (RIL)
population (n = 94) derived from (B. napus × B. rapa
turnip) × B. napus interspecific cross and following
genome-wide association study (GWAS) approach and
using software program TASSEL 4.0

| | | | | | Physical |
|---|---|---|---|---|---|
| | | | | | position |
| Chromo- | | | R² | | (Mb) on B. rapa |
| some | Marker | LOD | (%) | Add | cv. Chiffu |
| A02 | SSR_A2_5157 | 5.61 | 18.98 | −21.6 | 0.81 |
| A02 | SSR_A2_5168 | 5.52 | 18.63 | −21.1 | 3.63 |
| A03 | SSR_A3_3109 | 4.55 | 17.87 | −9.9 | 1.78 |
| A03 | SSR_A3_3157 | 6.92 | 27.13 | −16.9 | 0.98 |
| A08 | SSR_A08_972 | 5.71 | 22.56 | −13.3 | 14.34 |
| A08 | SSR_A08_2879 | 6.17 | 21.15 | −24.7 | 16.21 |
| A08 | SSR_A08_2330 | 7.26 | 25.23 | −31.0 | 20.87 |
| A09 | SSR_A9_866 | 6.27 | 24.70 | −14.9 | 38.60 |
| A09 | SSR_A9_1006 | 4.44 | 17.41 | −9.6 | 43.84 |
| A09 | SSR_A9_1005 | 8.75 | 33.60 | −23.0 | 43.86 |
| A09 | SSR_A9_1021 | 8.85 | 33.91 | −23.4 | 42.00 |
| A09 | SSR_A9_1014 | 4.69 | 18.43 | −10.3 | 44.41 |
| A09 | SSR_A9_771 | 5.98 | 23.59 | −14.0 | 37.93 |
| A09 | SSR_A9_1004 | 4.42 | 17.31 | −9.5 | 43.92 |
| A09 | SSR_A9_91 | 14.42 | 50.15 | −45.8 | 41.55 |
| A09 | SSR_A9_786 | 16.77 | 55.74 | −57.3 | 36.23 |
| A09 | SSR_A9_770 | 8.89 | 34.06 | −23.5 | 38.01 |
| A09 | SSR_A9_330 | 5.47 | 21.59 | −12.5 | 20.25 |
| A09 | SSR_A9_1029 | 5.84 | 23.04 | −13.6 | 40.94 |
| A09 | SSR_A9_802 | 4.30 | 16.81 | −9.2 | 31.46 |
| A09 | SSR_A9_878 | 7.19 | 28.12 | −17.8 | 36.85 |
| A09 | SSR_A9_865 | 4.74 | 18.66 | −10.4 | 39.99 |

Note.
LOD = logarithm of the odd; R² = phenotypic variation explained by the QTL.

Add=Estimated additive effect of the QTL; negative sign indicates ‘B. rapa turnip’ allele reduces disease severity index (DSI), i.e. contribute to resistance.

Genotyping the RIL Population for Association of the Markers with Resistance

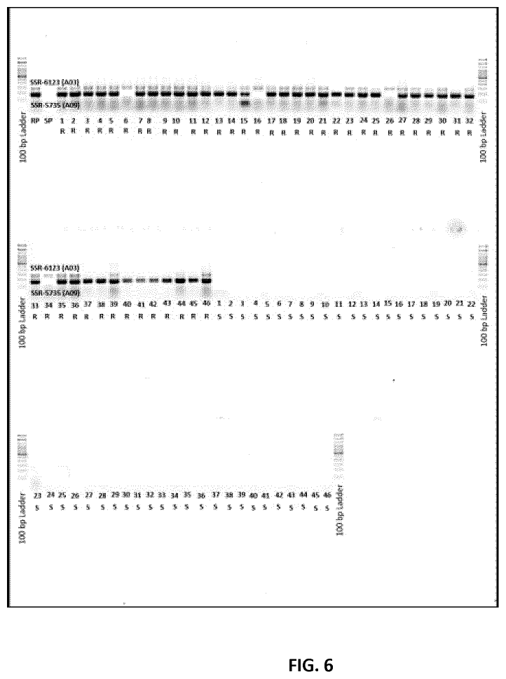

Of the total 107 SSR and 26 AS-SNP markers from the 1 to 10 Mb region of A03 and 37 to 40 Mb region of A09, 43 SSR (17 from A03 and 26 from A09) and 19 AS-SNP (from A09) markers showed polymorphism between the resistant (ECD 01 cv. Debra) and susceptible (A04-73NA) parents. The polymorphic markers were used to genotype the 46 resistant and 46 susceptible RILs for co-segregation of the markers with resistance to P. brassicae pathotype 3A. None of the markers individually showed strong linkage association with the resistance. However, a joint analysis of the markers from A03 and A09 QTL showed an excellent co-segregation with the clubroot resistance phenotype. For example, while using the marker SSR-5735 from 37.7 Mb region of A09 and the marker SSR-6123 from 1.6 Mb region of A03 QTL in a single PCR reaction, the genotypic data showed that all resistant lines carried the resistant parent allele of at least one of the two loci, while the susceptible lines were lacking the resistant parent allele (FIG. 6).

FIG. 6 depicts an agarose gel picture of 92 Brassica napus recombinant-inbred lines (RIL) lines genotyped with simple sequence repeat (SSR) markers SSR-5735 from A09 (37.7 Mb region) and SSR-6123 (1.6 Mb region) from A03 QTL; Invitrogen 100 bp Ladder (Thermo Fisher) is also included in the gel picture. The band of about 200 bp size, generated by SSR-5735 marker of the A09 locus, and the band of about 290 bp size, generated by SSR-6123 marker of the A03 locus, showed co-segregation with clubroot resistance. RP signifies the resistant parent ECD 01 (Brassica rapa var. rapifera); SP signifies the susceptible parent B. napus spring canola line A04-73NA; R and S signifies the resistant and susceptible lines of the RIL population, respectively.

### SNP Genotyping and QTL Mapping

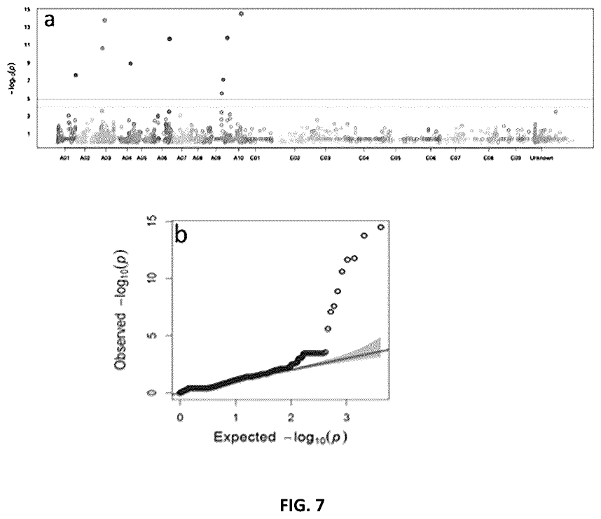

Genome-wide association mapping by using 4144 SNPs identified nine SNPs with LOD score of >5.6 from the chromosomes A01, A03, A04, A06, A09 and A10 to be associated with resistance to P. brassicae pathotype 3A (FIG. 7a); however, multiple SNPs from a genomic region could be detected only from the A03 and A09 chromosomes. Therefore, the two genomic regions of these two chromosomes were considered to be the reliable QTL. These two QTL included two SNPs from 10.9-14.5 Mb region of A03 and four SNPs from 28.7-37.3 Mb region of A09 (Table 8). These six SNPs were detected with LOD score of 5.57 to 13.75, and their genetic effect varied from 15.8 to 23.7% disease severity index (DSI); in all cases, allele of the resistant parent ECD 01 reduced DSI. The minor allele frequency (MAF) of these SNPs varied from 0.298 (29.8%) to 0.383 (38.3%) demonstrating the reliability of these SNPs to be associated with the resistance. The Quantile-Quantile (QQ) plot analysis also confirmed the reliability of the results obtained by using the software program BLINK. The Q-Q plots showed a large divergence for the p-values of the observed vs. expected LOD [−log 10(p)] distributions from the ‘tail’ indicating that these SNPs to be associated with clubroot resistance (FIG. 7b).

TABLE 8
List of the significant SNP markers identified through genome wide association study (GWAS) using 92 Brassica napus
recombinant inbred lines (RILs) derived from (B. napus × B. rapa turnip cv. Debra ECD 01) × B. napus
interspecific cross associated with resistance to Plasmodiophora brassicae pathotype 3A.

| | | | | | | | | LOD | Source of |
|---|---|---|---|---|---|---|---|---|---|
| SNP | Alleles | Chromosome | Position | MAF | P. value | R-square | Effect | score | major allele |
| Bn-A03-p10547280 | G [symbol] A | A03 | 10854885 | 0.383 | [symbol] .49E−11 | 6.540 | [symbol] 3.29 | 10.60 | ECD 01 |
| Bn-A03-p14015779 | A [symbol] C | A03 | 14501063 | 0.346 | 1.80E−14 | 7.331 | [symbol] 3.67 | 13.75 | ECD 01 |
| Bn-A09-p [symbol] 37 [symbol] 4102 | G [symbol] A | A09 | [symbol] 8692574 | 0.309 | [symbol] .70E−06 | 4.109 | 15.80 | 5.57 | ECD 01 |
| Bn-A09-p [symbol] 37 [symbol] 4105 | C [symbol] T | A09 | [symbol] 8692577 | 0.309 | [symbol] .70E−06 | 4.108 | 15.80 | 5.57 | ECD 01 |
| Bn-A09-p [symbol] 5575397 | C [symbol] A | A09 | 30991601 | 0. [symbol] 98 | 8.38E−08 | 6.919 | 18.61 | 7.08 | ECD 01 |
| Bn-A09-p31611279 | C [symbol] T | A09 | 37344900 | 0.378 | 1.69E−12 | 8.562 | 18.12 | 11.77 | ECD 01 |

[symbol]

indicates data missing or illegible when filed

### Epistasis Analysis of the Clubroot Resistance Loci

Based on the results from WGRS-based and SNP-based QTL mapping approaches, a digenic epistasis analysis was carried out for resistance to pathotype 3A. This analysis showed that the A02 QTL (about 0.81 to 2.63 Mb position) showed an epistatic interaction with the A03 QTL (about 2.9 to 5.3 Mb position) with additive×additive interaction effect of about 8.45% reduced DSI (Table 9). The A03 QTL also showed an epistatic interaction with the A09 QTL with additive×additive interaction effect of about 4.6% reduced DSI. Thus, interaction of these QTL further reduced the DSI, i.e. contributed to the resistance to P. brassicae pathotype 3A.

TABLE 9
Epistatic effect of the quantitative trait loci (QTL) of the A02, A03 and A09 chromosomes for resistance
to Plasmodiophora brassicae pathotype 3A detected in a Brassica napus recombinant-inbred (RIL) population
derived from (B. napus × B. rapa turnip ECD 01 cv. Debra) × B. napus interspecific
cross; analysis carried out using inclusive composite interval mapping (ICIM-Epi) method.

| | | Position | | Position | | | Position |
|---|---|---|---|---|---|---|---|
| | | of the | | of the | | | of the |
| | Left | Left | Right | Right | | Left | Left |
| Chromosome1 | Marker1 | Marker1 | Marker1 | Marker1 | Chromosome2 | Marker2 | Marker2 |
| A02 | SSRid_A2_5157 | 0.81 Mb | SSRid_A2_5215 | 2.68 Mb | A03 | SSRid_A3_6132 | 2.9 Mb |
| A03 | SSRid_A3_6132 | 2.9 Mb | SSRid_A3_6122 | 1.58 Mb | A09 | SSRid_A9_1 [symbol] 27 | <0.9 Mb |

| | | Position | | | | | |
|---|---|---|---|---|---|---|---|
| | | of the | | | | | |
| | Right | Right | | PVE | | | Add × |
| Chromosome1 | Marker2 | Marker2 | LO [symbol] | (%) | Add1 | Add2 | Add |
| A02 | SSRid_A3_6142 | 5.3 Mb | 2.60 | 14.73 | <.51 | 11.79 | 8.45 |
| A03 | SSRid_A9_1 [symbol] 12 | <4.4 Mb | 3.07 | 13.19 | 8.08 | 6.13 | <.60 |

Note:
LOD, Logarithm of odd; PVE, phenotypic variation explained by the QTL; Add1, estimated additive effect of the QTL of Chromosome 1; Add2, estimated additive effect of the QTL of Chromosome 2; Add × Add, estimated additive by additive interaction effect of two QTL. [symbol]
indicates data missing or illegible when filed

### General Discussion on QTL Mapping Based on Results Herein

While taking the WGRS-based and SNP-based QTL mapping results together, it is apparent that at least two major loci from A03 and A09 chromosomes contributed to the resistance to P. brassicae pathotype 3A through direct effect as well as through epistatic interaction. WGRS-based QTL mapping identified these two loci at about 1.7-10.2 Mb of A03 and at about 38.01-41.5 Mb of A09, while the SNP-based QTL mapping identified these two loci at about 10.9-14.5 Mb region of A03 and 28.7-37.3 Mb region of A09. Thus, both approaches detected clubroot resistance loci on the A03 and A09 chromosomes; however, at slightly different positions. Based on this, it can be postulated that the 1.7 to 14.5 Mb region of A03 and 28.7 to 41.5 Mb region of A09 carry this resistance. However, when we tested 15 polymorphic markers from 1.58 to 9.51 Mb region of A03 and 12 polymorphic markers from 37.7 to 40.2 Mb region of A09, we found 15.2 to 48.6% recombination for the A03 markers and 8.1 to 39.1% recombination for the A09 markers with the resistance. The A03 marker which showed the lowest recombination (15.2%) is located at 5.3 Mb region, while the markers from its upstream region (i.e. towards the lower Mb position), such as from the 1.6 Mb region, showed a greater recombination (up to 24%). In case of the A09 QTL, the marker from 38.4 Mb region showed the lowest recombination (8.1%) while the marker from its downstream region (i.e. towards the higher Mb position) (40.2 Mb) showed a greater (17.4%) recombination. This indicated that these markers are located away from the clubroot resistance genes; the A03 gene might be located downstream of the 5.3 Mb region and the A09 gene might be located upstream of the 38.4 Mb region. Based on this, the marker analysis supports the results obtained from SNP-based QTL mapping that the 10 to 15 Mb region of A03 and 28 to 37 Mb region of A09 carry the clubroot resistance genes.

In addition to the above-mentioned two QTL, the A02 and A08 QTL also played a role in clubroot resistance; however, these two loci could not be detected following all approaches of QTL mapping.

### Comparison of Presently Disclosed Loci to Known Loci

Previously, QTL or major loci conferring resistance to P. brassicae have been reported at 18.50-26.35 Mb region of A02, 15.32-26.81 Mb region of A03, and 11.34-21.75 Mb region of A08 (for review, see Hasan et al. 2021; Yu et al. 2017). QTL mapping of clubroot resistance using a B. napus mapping population carrying clubroot resistance of B. rapa ECD 04 has been performed and showed discrete segregation for resistance (variation from resistant to susceptible) to six P. brassicae isolates, (other than the isolate ‘Korporal’), and based on this, Werner et al. (2008) reported two major QTL on A03 and A08. In regard to resistance to the isolate ‘Korporal’, this mapping population showed a continuous variation from partial resistance to susceptibility, and based on this data they reported a minor QTL on A09 exerting an additive effect only 4.9% DSI; physical position of this QTL could not be found in this publication. Thus, it is apparent that no major clubroot resistance locus has so far been reported on chromosome A09; in this regard, the presently disclosed A09 locus conferring resistance to P. brassicae pathotype 3A is novel. Of the 47 resistant lines of the mapping population, most of the lines also carry resistance to pathotype 3H; therefore, the presently disclosed A09 locus introgressed into canola may also play a role in resistance to pathotype 3H.

The presently disclosed A02 locus is located at about 1-4 Mb position of the B. rapa cv. Chiffu v3.0 reference genome, compared to previously known locus at 18.50-22.10 Mb region (Yu et al. 2017). In this regard, the presently disclosed A02 locus is different from previously reported locus. Others (Sakamoto et al. 2008; Werner et al. 2008) have shown clubroot resistance loci on A02; however, physical position of these loci cannot be confirmed.

The presently disclosed A03 locus is located at about 1-2 Mb region (FIGS. 5 and 6; Table 6, 7, and 8), while researchers previously reported clubroot resistance loci from 15.32-26.81 Mb region of A03 (for review, see Hasan et al. 2021).

P. brassicae Pathotype Resistance

### Evaluation of the Near-Isogenic Lines (NILs) for Resistance to Different Pathotypes

In some embodiments, near-isogenic clubroot resistant and susceptible B. napus lines may be developed by crossing one of the pathotype 3A resistant lines derived from (B. napus x B. rapa turnip)×B. napus interspecific cross with the B. napus parent A04-73NA. For this, the F1 plants may be recurrently backcrossed to A04-73NA for four times and the resulting BC4 plants may be self-pollinated for two generations. In each backcross generation, plants exhibiting resistance to pathotype 3H may be backcrossed to the recurrent parent A04-73NA. The resulting BC4F3 lines showing resistance or susceptibility to pathotype 3H may be evaluated for resistance to 10 P. brassicae pathotypes, viz. 3H, 2B, 3A, 3D, 5G, 5X, 5L, 5O, 8E and 8P. In preferred embodiments, the resistant lines may be resistant to all 10 pathotypes (DSI <30%); conversely, susceptible lines may be susceptible to all pathotypes (DSI >80%) (Table 10).

TABLE 10
Evaluation of the near-isogenic lines (NILs) of Brassica napus for resistance to 10
Plasmodiophora brassicae pathotypes. The resistant and susceptible NILs were developed
by using clubroot resistance of the Brassica rapa turnip cv. Debra (ECD 01). For
resistance to different pathotypes, disease severity index (DSI, %) values are presented,
where DSI < 30% is considered as resistant and >70% considered as susceptible.

| NILS | 3H | 2B | 3A | 3D | 5G | 5X | 5L | 5O | 8E | 8P |
|---|---|---|---|---|---|---|---|---|---|---|
| 1CA2512.343-B4F3-21 | 8 | 0 | 0 | 0 | 0 | 4 | 0 | 4 | 0 | 0 |
| 1CA2512.348-B4F3-21 | 8 | 0 | 0 | 0 | 0 | 0 | 0 | 0 | 0 | 0 |
| 1CA2512.349-B4F3-21 | 13 | 0 | 0 | 0 | 0 | 3 | 0 | 4 | 0 | 0 |
| 1CA2512.351-B4F3-21 | 8 | 0 | 0 | 0 | 0 | 3 | 0 | 0 | 0 | 0 |
| 1CA2512.334-B4F3-21 | 100 | 100 | 81 | 100 | 100 | 100 | 100 | 100 | 100 | 100 |
| 1CA2512.341-B4F3-21 | 100 | 100 | 86 | 100 | 99 | 100 | 100 | 100 | 100 | 96 |
| 1CA2512.342-B4F3-21 | 100 | 100 | 90 | 100 | 100 | 100 | 100 | 100 | 100 | 93 |
| 1CA2512.346-B4F3-21 | 100 | 100 | 83 | 100 | 96 | 100 | 100 | 100 | 100 | 100 |
| 1CA2512.338-B4F3-21 | 100 | 100 | 94 | 100 | 100 | 100 | 100 | 100 | 100 | 96 |
| 1CA2512.339-B4F3-21 | 100 | 100 | 92 | 100 | 98 | 100 | 100 | 100 | 100 | 100 |
| 1CA2512.340-B4F3-21 | 100 | 100 | 92 | 100 | 96 | 100 | 100 | 100 | 99 | 94 |

### Transcriptome Analysis of the NILs and Molecular Marker Development

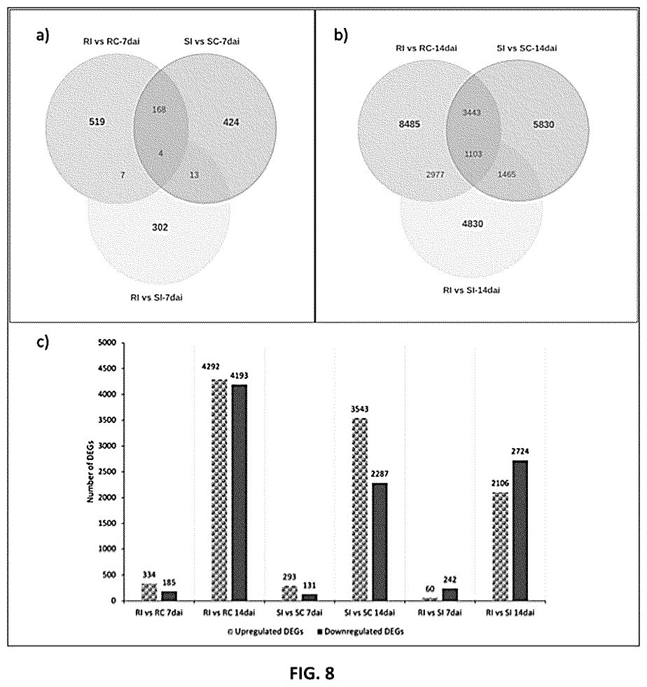

Transcriptome (RNA-seq) analysis was carried out using the above-mentioned clubroot resistant and susceptible NILs. A total 1.01 billion clean reads were obtained from RNA-seq of which about 88% of the reads were mapped to the B. napus reference genome Darmor-bzh v4.1 and most of them (84.03%) mapped to one position in the genome. Of the total reads from all samples and time points, 106,682 reads were annotated where 20,390 genes were found to be differentially expressed due to the difference in genotype and response to pathogen infection. From the RI (resistant inoculated) vs. RC (resistant control) comparison, 519 DEGs (differentially expressed genes) were identified in the CR-NILs at 7 dai, while about 16-fold greater number (8,485) of DEGs were identified at 14 dai (FIG. 8a, 8b). From the SI (susceptible inoculated) vs. SC (susceptible control) comparison, 424 and 5,830 DEGs were identified at 7- and 14 dai, respectively (FIG. 8a, 8b). Thus, a greater number of genes showed differential expression in the CR-NILs as compared to the CS-NILs. In the RI (resistant inoculated) vs. SI (susceptible inoculated) comparison, 302 DEGs (60 upregulated, 242 downregulated) were identified at 7 dai and 4,830 DEGs (2106 upregulated, 2724 downregulated) were identified at 14 dai (FIG. 8a, 8b, 8c). Based on the above-mentioned three comparisons, 7 and 13 genes were found to be uniquely expressed, respectively, in the CR- and CS-NILs at 7 dai, while 2,977 and 1,465 genes were uniquely expressed, respectively, in the CR- and CS-NILs at 14 dai (FIG. 8a, 8b).

FIG. 8. Venn diagrams showing differentially expressed genes (DEGs) (p-value ≥0.05) in clubroot resistant and susceptible Brassica napus near isogenic lines (NILs) due to infection by Plasmodiophora brassicae pathotype 3H at (a) 7 days after inoculation (dai) and (b) 14 dai from three comparisons of the resistant (R) and susceptible (S) near-isogenic lines (NILs) with or without inoculation. The overlapping regions shows the number of DEGs detected from more than one comparison. (c) Histogram of the number of upregulated and downregulated DEGs detected at 7- and 14 dai from three comparisons: Resistant inoculated (RI) NILs against its control (RC), susceptible inoculated (SI) NILs against its control (SC), and resistant inoculated (RI) NILs against susceptible inoculated (SI) NILs.

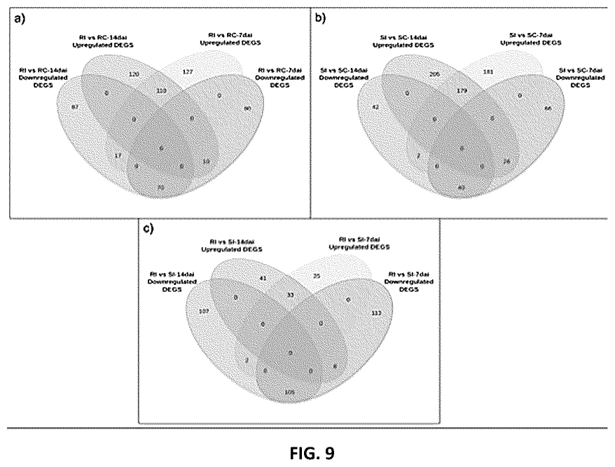

In case of the RI vs. RC comparison, a total of 207 DEGs could be detected at both 7- and 14 dai, where 110 DEGs were upregulated, 70 downregulated and the remaining DEGs showed contrasting expression at these two time points (FIG. 9a). For the SI vs. SC comparison, 247 DEGs detected at both 7- and 14 dai, where 179 and 40 DEGs showed upregulation and downregulation, respectively, and 28 DEGs showed opposite expression at these two time points (FIG. 9b). In case of the RI vs. SI comparison, 154 DEGs detected at both 7- and 14 dai; among these 33 were upregulated and 105 downregulated, while the remaining 16 showed opposite expression at these two time points (FIG. 9c).

FIG. 9 depicts a Venn diagrams representing the number of upregulated and downregulated differentially expressed genes (DEGs) in Brassica napus near isogenic lines (NILs) due to infection by Plasmodiophora brassicae pathotype 3H at 7 days after inoculation (dai) and 14 days after inoculation (dai) from three comparisons of the near-isogenic lines (NILs): (a) resistant inoculated (RI) NILs against its control (RC), (b) susceptible inoculated (SI) NILs against its control (SC), and (c) resistant inoculated (RI) NILs against susceptible inoculated (SI) NILs.

To gain a better understanding of the biological mechanisms responsive to P. brassicae infection, GO enrichment analysis was performed on the DEGs detected based on RI vs. RC, SI vs. SC and RI vs. SI comparisons. The GO terms specific to the CR-NILs included response to oxidative stress (GO:0006979), defense response (GO:0006952), peroxidase activity (GO:0004601), oxidoreductase activity (GO:0016684), response to external biotic stimulus (GO:0043207), response to other organism (GO:0051707), and defense response to other organism (GO:0098542).

To get a deeper insight into the biological significance of gene function, the DEGs were analyzed by KEGG. The top 20 enriched KEGG pathways included “ath00940: phenylpropanoid biosynthesis”, “ath00500: starch and sucrose metabolism”, “ath04075: plant hormone signal transduction”, “ath01200: carbon metabolism” and “ath04626: plant-pathogen interaction”.

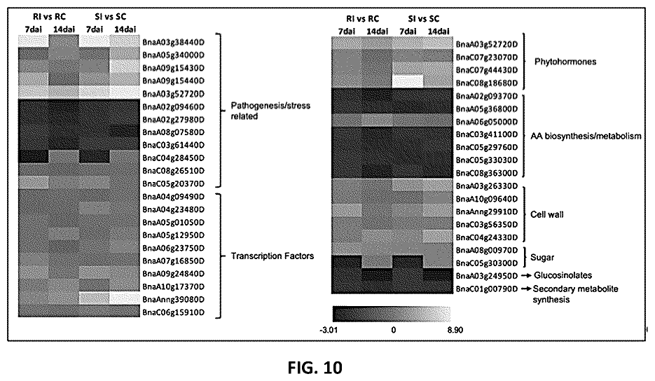

From the RI vs. RC and SI vs. SC comparisons, we found 50 common DEGs related to pathogenesis and stress response (12), transcription factors (10), amino acid biosynthesis and metabolism (seven), cell wall modifications (five), phytohormone signaling (four), and sugar (two), glucosinolates (one) and secondary metabolite synthesis (one) (FIG. 10).

FIG. 10 depicts heatmaps representing the expression profiles of the differentially expressed genes (DEGs) related to different biological pathways in the clubroot resistant and susceptible Brassica napus near isogenic lines (NILs) (CR-NILs and CS-NILs) inoculated with Plasmodiophora brassicae pathotype 3H with respect to their controls at 7- and 14-days after inoculation (dai). RI=inoculated resistant NILs; RC=uninoculated (control) resistant NILs; SI=inoculated susceptible NILs; SC=uninoculated (control) susceptible NILs.

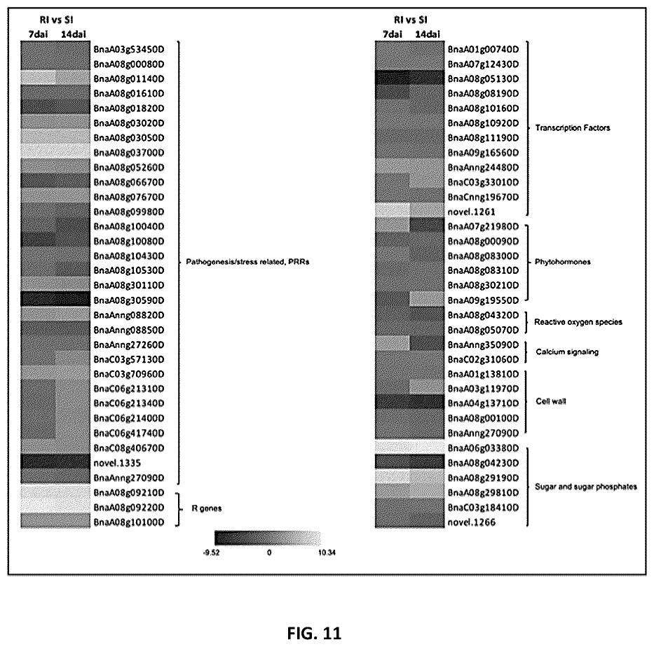

While comparing the RI vs. SI, we identified a total of 148 DEGs at both time points (7 dai and 14 dai). This included 30 DEGs related to pathogenesis and stress response, three R genes, 12 transcription factors, seven amino acid biosynthesis and metabolism, two reactive oxygen species, two Ca-mediated signalling, five cell wall modifications, six phytohormone signaling, and six sugar and sugar phosphates (FIG. 11).

FIG. 11 depicts heatmaps representing the expression profiles of the differentially expressed genes (DEGs) related to different biological pathways in the clubroot resistant and susceptible Brassica napus near isogenic lines (NILs) (CR-NILs and CS-NILs) inoculated with Plasmodiophora brassicae pathotype 3H at 7- and 14-days after inoculation (dai). RI=inoculated resistant NILs; SI=inoculated susceptible NILs.

Genotyping the RILs for Association of the Markers with Resistance

The A03 and A09 QTL regions were searched manually for DEGs to be involved in disease resistance, and 47 DEGs from about 1 to 10 Mb region of A03 and 80 DEGs from about 37 to 43 Mb region of A09 were identified. Based on the DEGs from the A03 region, 40 SSR markers were designed of which 42.5% markers (17/40) showed polymorphism between the parents ECD 01 (cv. Debra) and A04-73NA. However, genotyping of 23 resistant and 23 susceptible RILs with the polymorphic markers failed to demonstrate strong linkage association of these markers with resistance; minimum recombination between the markers and resistance was about 15%, i.e. the closest marker was about 15 cM away from the resistance locus. The marker showing the least recombination (about 15%) was from about 7.5 Mb region. This demonstrated that the A03 QTL might be located downstream (i.e. towards the higher Mb position) of this location, i.e. about 10-15 Mb region detected by SNP-based QTL mapping

In case of the A09 chromosome, considering the DEGs as well as the SNP genotyping result we designed 30 SSR markers based on 25 DEGs from 27-34 Mb region; among these, seven markers showed polymorphism between the parents. We used the polymorphic markers to genotype the entire mapping population which included 46 resistant and 46 susceptible RILs. Recombination frequency of the markers with clubroot resistance varied from 1.08% to 21.46%. The marker SSR-6115, which was designed based on the gene BnaA09g48550D (homologous to Respiratory Burst Oxidase Homolog protein E or RBOHE), showed the strongest linkage association with least (1.08%) recombination frequency (FIG. 12); this gene is located at 30.42 Mb position of A09. This suggests that the A09 QTL is located at about 30 Mb region of this chromosome. This further supports the results from the WGRS based marker analysis where we found a greater recombination (17.4%) for the marker from 40.2 Mb as compared to the marker from 38.4 Mb region (8.1% recombination) demonstrating that the clubroot resistance is located upstream (i.e. towards the lower Mb position) of the 38.4 Mb region, i.e. towards the 30 Mb region. To note here that the SNP-based genotyping identified four SNPs from A09 chromosome associated with clubroot resistance where three of them located at 28.69 to 30.99 Mb region (Table 8); this further supports that this QTL is located at about 30 Mb region of A09.

FIG. 12 depicts an agarose gel picture of 92 Brassica napus recombinant-inbred lines (RIL) lines genotyped with simple sequence repeat (SSR) marker SSR-6115 from A09 chromosome (30.42 Mb region); Invitrogen 100 bp Ladder (Thermo Fisher) is also included in the gel picture. The band of about 300 bp size, generated by the SSR-6115 marker of the A09 locus, showed co-segregation with clubroot resistance. RP signifies the resistant parent ECD 01 (Brassica rapa var. rapifera); SP signifies the susceptible parent B. napus spring canola line A04-73NA; R and S signifies the resistant and susceptible lines of the RIL population, respectively.

### Proteome Analysis of the NILs

A total of 10,998 proteins were identified across the genotypes, treatments and time points of which 6,757 were significantly (q<0.05) differentially accumulated (DAPs). The total number of DAPs detected from the RI vs. RC, SI vs. SC and RI vs. SI comparisons was 2353, 4274 and 4361, respectively (FIG. 13). It was found that the number of DAPs increased by 1.5 to 5.5 times as the infection progressed from 7- to 21 dpi (FIG. 13).

FIG. 13 depicts differentially accumulated proteins (DAPs) in roots of the clubroot resistant (CR) and susceptible (CS) Brassica napus near-isogenic lines (NILs) inoculated with Plasmodiophora brassicae pathotype 3H. RI=inoculated resistant NILs; RC=uninoculated (control) resistant NILs; SI=inoculated susceptible NILs; SC=uninoculated (control) susceptible NILs; dpi=days post inoculation.

To identify the putative proteins associated with resistance against P. brassicae, the gene ontology (GO) enrichment analysis was performed on the DAPs from the RI vs. RC, SI vs. SC, and RI vs. SI comparisons. The top significant GO terms included the followings: Response to external stimulus (GO:0009605), biological process involved in interspecies interaction between organisms (GO:0044419), response to other organism (GO:0051707), response to external biotic stimulus (GO:0043207), response to biotic stimulus (GO:0009607), oxidoreductase activity (GO:0016491), antioxidant activity (GO:0016209), and peroxidase activity (GO:0004601). The KEGG pathway enrichment analysis of data from the RI vs. RC comparison identified 377 significantly enriched (FDR <0.05) DAPs belonging to three pathways, viz. protein processing in endoplasmic reticulum (44 DAPs), metabolic pathways (304 DAPs), and amino sugar and nucleotide sugar metabolism (29 DAPs). From the SI vs. SC comparison, 1460 significantly enriched (FDR <0.05) DAPs were identified and these DAPs belonged to 16 pathways; among these two pathways viz., ribosome and ribosome biogenesis in eukaryotes were unique for this comparison. The RI vs. SI comparison detected 1318 significantly enriched (FDR <0.05) DAPs, which belonged to 20 pathways; among them the pathways, viz. sulfur, glutathione, pyruvate, and butanoate metabolism as well as starch and sucrose metabolism, valine, leucine and isoleucine degradation and citrate cycle were unique to the CR-NILs.

On the basis of the functional enrichment analysis and the pattern of accumulation, the putative DAPs involved in clubroot resistance included the ones associated with general stress response, calcium-mediated signalling, phytohormone-mediated signalling, reactive oxygen species, and lipid and glucosinolate metabolism. We found an increased abundance of four LRR-containing proteins (receptor-like serine/threonine-protein kinases) in the inoculated CR-NILs for the perception of signal. This led to the increased abundance of three ubiquitin-dependent proteins, viz. 26S proteasome non-ATPase regulatory subunit 2 homolog B (RPN1B, BnaA01T0006100WE), E3 ubiquitin protein ligase (RGLG2, BnaA07T0290200WE) and ubiquitin carboxyl-terminal hydrolase 12 (UBP12, BnaC02T0487200WE) in the CR-NILs suggesting the role of ubiquitination-mediated signalling pathway in clubroot resistance. The proteins involved in Ca-signalling (CML49, RALF22, RALF1, IQD1) and ROS-mediated signalling (peroxidases, catalase, GST U12, CLPR4) showed an increased accumulation in the CR-NILs at least at one time point. We also identified putative proteins related to R genes (EDR2-like, YLS9, SDR1) showing an increased abundance in the CR-NILs but a decreased abundance in the CS-NILs implying that they might be involved in P. brassicae-B. napus interaction and playing a role in clubroot resistance. We identified the proteins orthologous to NSP2, NSP5 and AOP1 showing an increased abundance in the inoculated CR-NILs. These proteins are involved in glucosinolate metabolism and hence might be playing a role in plant defense response.

### General Comments Based on Transcriptome and Proteome Analysis

The NILs used in the RNA-seq and proteomics experiments were resistant to more than 10 P. brassicae pathotypes including the ones that evolved recently. We witnessed an intense defense response in the resistant NILs as compared to the susceptible NILs. On the basis of the results from these experiments, we could provide information on the role of genes associated with phytohormone signaling, disease resistance, calcium influx and ROS, WRKY TFs and glucosinolates biosynthesis in clubroot resistance in B. napus. Functional validation of these genes would extend our knowledge on the molecular mechanisms underlying clubroot resistance in B. napus as well as to develop gene-based molecular markers for use in breeding.

It will be appreciated by those skilled in the art that various changes and modifications can be made to these embodiments without changing or departing from their scope, intent, or functionality. These terms and expressions used in the preceding specification have been used herein as terms of description and not of limitation, and there is no intention in the use of such terms and expressions of excluding equivalents of the features shown and the described portions thereof.

Method of the invention are conveniently practiced by providing the compounds and/or compositions used in such method in the form of a kit. Such kit preferably contains the composition. Such a kit preferably contains instructions for the use thereof.

The embodiments described herein are intended to be examples only. Alterations, modifications and variations can be effected to the particular embodiments by those of skill in the art. The scope of the claims should not be limited by the particular embodiments set forth herein, but should be construed in a manner consistent with the specification as a whole.

### Examples

In some examples, there is provided an open-pollinated or synthetic oilseed Brassica napus canola or other types of oilseed B. napus (e.g. high erucic acid) cultivar.

In some examples, there is provided a hybrid oilseed Brassica napus canola or other types of oilseed B. napus (e.g. high erucic acid) cultivar.

In some examples, there is provided a female and male parent lines (A, B, and C or R lines) of hybrid oilseed Brassica napus canola or other types of oilseed B. napus (e.g. high erucic acid) cultivar. Breeders first develop parent lines and they use these lines to develop a hybrid cultivar.

In some examples, there is provided an open-pollinated or synthetic vegetable or fodder Brassica napus cultivar, such as rutabaga or swede and forage rape.

In some examples, there is provided a hybrid vegetable or fodder Brassica napus cultivar, such as rutabaga or swede and forage rape.

In some examples, there is provided a female and male parent lines (A, B, and C or R lines) of hybrid vegetable or fodder Brassica napus cultivar, such as rutabaga or swede and forage rape.

In some examples, there is provided a transfer this gene into the A genome of oilseed or vegetable or fodder Brassica rapa, and by using this develop an open-pollinated or synthetic or hybrid Brassica rapa cultivar (can be canola quality or non-canola quality type such as high-erucic).

In some examples, the B. rapa carrying this gene (transferred as above) can be used to develop oilseed or vegetable or fodder Brassica napus cultivar. This can be either an open-pollinated or synthetic or hybrid cultivar.

In some examples, there is provided a transfer this gene in the C genome of Brassica oleracea, and by using this develop an open-pollinated or synthetic or hybrid Brassica oleracea cultivar.

In some examples, the B. oleracea carrying this gene (transferred as above) can be used to develop oilseed or vegetable or fodder Brassica napus cultivar. This can be either an open-pollinated or synthetic or hybrid cultivar.

### REFERENCES

Abe A, Kosugi S, Yoshida K, Natsume S, Takagi H, Kanzaki H, Matsumura H, Yoshida K, Mitsuoka C, Tamiru M, Innan H, Cano L, Kamoun S, Terauchi R, 2012. Genome sequencing reveals agronomically important loci in rice using MutMap. Nature Biotechnology 30:174-178. https://doi.org/10.1038/nbt.2095
Attri R, Rahman H, 2018. Introgression of allelic diversity from genetically distinct variants of Brassica rapa into Brassica napus canola and inheritance of the B. rapa alleles. Crop & Pasture Science 69:94-106. doi.org/10.1071/CP17193
Bolger A M, Lohse M, Usadel B, 2014. Trimmomatic: A flexible trimmer for Illumina sequence data. Bioinformatics 30:2114-2120. https://doi.org/10.1093/bioinformatics/btu170.
Boskovic J, Boskovic M, Babovic M, Jerkovic Z, Pesic V, 2001. Pyramiding strategy for durable resistance to wheat leaf rust pathogen. In Developments in Plant Breeding. 9th ed. Edited by Z. Bedo and L. Lang. Springer, Dordrecht. pp. 337-343. doi:10.1007/978-94-017-3674-9_43
Bradbury P J, Zhang Z, Kroon D E, Casstevens T M, Ramdoss Y, Buckler E S, 2007. ASSEL: Software for association mapping of complex traits in diverse samples. Bioinformatics 23: 2633-2635. https://doi.org/10.1093/bioinformatics/btm308
Buels R, Yao E, Diesh C M, Hayes R D, Munoz-Torres M, Helt G, Goodstein D M, Elsik C G, Lewis S E, Stein L, Holmes I H, 2016. JBrowse: A dynamic web platform for genome visualization and analysis. Genome Biology 17:66. https://doi.org/10.1186/s13059-016-0924-1
Chalhoub B, Denoeud F, Liu S, Parkin I A P et al., 2014. Early allopolyploid evolution in the post-Neolithic Brassica napus oilseed genome. Science 345:950-953. https://doi.org/10.1126/science.1253435.
Chu M, Yu F, Falk K C, Liu X, Zhang X, Chang A, Peng G, 2013. Identification of the clubroot resistance gene Rpb1 and introgression of the resistance gene into canola breeding lines using a marker-assisted selection approach. Acta Horticulturae 1005:599-605. DOI:10.17660/ActaHortic.2013.1005.74.
Conesa A, Gotz S, Garcia-Gomez J M, Terol J, Talon M, Robles M, 2005. Blast2Go: a universal tool for annotation, visualization and analysis in functional genomics research. Bioinformatics 21:3674-3676. Doi: 10.1093/bioinformatics/bti610
Dixon G R, 2009. The occurrence and economic impact of Plasmodiophora brassicae and clubroot disease. Journal of Plant Growth Regulation 28:194-202. DOI:10.1007/s00344-009-9090-y.
Ellis J G, Lagudah E S, Spielmeyer W, Dodds P N, 2014. The past, present and future of breeding rust resistant wheat. Frontiers in Plant Science 5:641. doi:10.3389/fpls.2014.00641. PMID:25505474.
Farid M, Yang R C, Kebede B, Rahman H, 2020. Evaluation of Brassica oleracea accessions for resistance to Plasmodiophora brassicae and identification of genomic regions associated with resistance. Genome 63:91-101. https://doi.org/10.1139/gen-2019-0098
Engqvist L G, 1994. Distribution of clubroot (Plasmodiophora brassicae Wor) in Sweden and the effect of infection on oil content of oilseed rape (Brassica napus L.). Journal of Swedish Seed Association 104:82-86.
Fredua-Agyeman R, Rahman H, 2016. Mapping of the clubroot disease resistance in spring Brassica napus canola introgressed from European winter canola cv. “Mendel.” Euphytica 211:201-213. DOI:10.1007/s10681-016-1730-2.
Fukuoka S, Saka N, Mizukami Y, Koga H, Yamanouchi U, Yoshioka Y et al., 2015. Gene pyramiding enhances durable blast disease resistance in rice. Scientific Reports 5:7773. doi:10.1038/srep07773
Hasan J, Megha S, Rahman H, 2021. Clubroot in Brassica: recent advances in genomics, breeding, and disease management. Genome 64:735-760. dx.doi.org/10.1139/gen-2020-0089
Hasan M J, Rahman H, 2016. Genetics and molecular mapping of resistance to Plasmodiophora brassicae pathotypes 2, 3, 5, 6, and 8 in rutabaga (Brassica napus var. napobrassica). Genome 59:805-815. doi:10.1139/gen-2016-0034.
Hasan M J, Shaikh R, Basu U, Rahman H, 2021. Mapping clubroot resistance of Brassica rapa introgressed into Brassica napus and development of molecular markers for the resistance. Crop Science 61:4112-4127. DOI:10.1002/csc2.20626.
Hasan M J, Strelkov S, Howard R, Rahman H, 2012. Screening of Brassica germplasm for resistance to Plasmodiophora brassicae pathotypes prevalent in Canada for broadening diversity in clubroot resistance. Canadian Journal of Plant Science 92:501-515. https://doi.org/10.4141/cjps2010-006
Hobson N, Rahman H, 2016. Genome-wide identification of SSR markers in the Brassica A genome and their utility in breeding. Canadian Journal of Plant Science 96: 808-818. https://doi.org/10.1139/cjps-2015-0250
Hirai M, 2006. Genetic analysis of clubroot resistance in Brassica crops. Breeding Science 56:223-229.
Hirani A H, Gao F, Liu J, Fu G, Wu C, Yuan Y, Li W, Hou J, Duncan R, Li G, 2016. Transferring clubroot resistance from Chinese cabbage (Brassica rapa) to canola (B. napus). Canadian Journal of Plant Pathology 38:82-90. DOI:10.1080/07060661.2016.1141799.
Huang M, Liu X, Zhou Y, Summers R M, Zhiwu Z, 2019. BLINK: a package for the next level of genome-wide association studies with both individuals and markers in the millions. Gigascience 8(2):giy154. doi: 10.1093/gigascience/giy154
Hurgobin B, Golicz A A, Bayer P E, Chan C-K K, Tirnaz S, Dolatabadian A, Schiessl S V, Samans B, Montenegro J D, Parkin I A P, Pires J C, Chalhoub B, King G J, Snowdon R, Batley J, Edwards D, 2018. Homoeologous exchange is a major cause of gene presence/absence variation in the amphidiploid Brassica napus. Plant Biotechnol J 16:1265-1274. https://doi.org/10.1111/pbi.12867
Iftikhar R, Wang X, Rahman H, 2018. Broadening the genetic base of Brassica napus canola by interspecific crosses with different variants of B. oleracea. Euphytica 214:133. https://doi.org/10.1007/s10681-018-2213-4.
Jiang H, Li Z, Liu J, Shen Z, Gao G, Zhang Q, He Y, 2019. Development and evaluation of improved lines with broad-spectrum resistance to rice blast using nine resistance genes. Rice 12:29. doi:10.1186/s12284-019-0292-z
Kageyama K, Asano T, 2009. Life cycle of Plasmodiophora brassicae. Journal of Plant Growth Regulation 28:203-211. doi:10.1007/s00344-009-9101-z.
Kanehisa M, Goto S, 2000. KEGG: kyoto encyclopedia of genes and genomes. Nucleic Acids Research 28(1):27-30. https://doi.org/10.1093/nar/28.1.27
Kaur K, Liu Y, Rahman H, 2022. Introgression of resistance to multiple pathotypes of Plasmodiophora brassicae from turnip (Brassica rapa ssp. rapifera) into spring B. napus canola. Agronomy 12:1225. https://doi.org/10.3390/agronomy12051225
Kim D, Langmead B, Salzberg S, 2015. HISAT: a fast spliced aligner with low memory requirements. Nat Methods 12:357-360. https://doi.org/10.1038/nmeth.3317
Koboldt D C, Zhang Q, Larson D E, Shen D, Mclellan M D, Lin L, Miller C A, Mardis E R, Ding L, Wilson R K, 2012. VarScan 2: Somatic mutation and copy number alteration discovery in cancer by exome sequencing. Genome Research 22:568-576. https://genome.cshlp.org/content/22/3/568
Kosambi D D, 1943. The estimation of map distances from recombination values. Annals of Eugenics 12:172-175. https://doi.org/10. 1111/j.1469-1809.1943.tb02321.x
Kotchoni S O, Gachomo E W, 2009. A rapid and hazardous reagent free protocol for genomic DNA extraction suitable for genetic studies in plants. Molecular Biology Reports 36:633-1636. https://doi.org/10.1007/s11033-008-9362-9
Kuginuki Y, Yoshikawa H, Hirai M, 1999. Variation in virulence of Plasmodiophora brassicae in Japan tested with clubroot-resistant cultivars of Chinese cabbage (Brassica rapa L. ssp. pekinensis). European Journal of Plant Pathology 105, 327-332. https://doi.org/10.1023/A:1008705413127.
Laroche A, Frick M, Graf R J, Larsen J, Laurie J D, 2019. Pyramiding disease resistance genes in elite winter wheat germplasm for Western Canada. Crop Journal 7:739-749. doi:10.1016/j.cj.2019.08.005.
Li H, Durbin R, 2009. Fast and accurate short read alignment with Burrows-Wheeler transform. Bioinformatics 25:1754-1760. https://doi.org/10.1093/bioinformatics/btp324.
Li H, Handsaker B, Wysoker A, Fennell T, Ruan J, Homer N, Marth G, Abecasis G, Durbin R, 2009. The Sequence Align-ment/Map format and SAMtools. Bioinformatics 25:2078-2079. https://doi.org/10.1093/bioinformatics/btp352
Li H, Ye G, Wang J, 2007. A modified algorithm for the improvement of composite interval mapping. Genetics 175:361-374. https://doi.org/10.1534/genetics.106. 066811
Lipka A E, Tian F, Wang Q, Peiffer J, Li M, Bradbury P J, Gore M A, Buckler E S, Zhang Z, 2012. GAPIT: genome association and prediction integrated tool. Bioinformatics, 28(18):2397-2399. doi:10.1093/bioinformatics/bts444. PMID:22796960.
Manichaikul A, Moon J Y, Sen S, Yandell B S, Broman K W, 2009. A model selection approach for the identification of quantitative trait loci in experimental crosses, allowing epistasis. Genetics 181:1077-1086. https://doi.org/10.1534/genetics.108.094565
Matsumoto E, Ueno H, Aruga D, Sakamoto K, Hayashida N, 2012. Accumulation of three clubroot resistance genes through marker-assisted selection in Chinese cabbage (Brassica rapa ssp. pekinensis). Journal of Japanese Society of Horticultural Science 81:184-190. doi:10.2503/jjshs1.81.184.
Mehta D, Scandola S, Uhrig R G, 2022. BoxCar and Library-free data-independent acquisition substantially improve the depth, range, and completeness of label-free quantitative proteomics. Anal Chem 94(2):793-802. https://doi.org/10.1021/acs.analchem.1c03338
Meng L, Li H, Zhang L, Wang J, 2015. QTL IciMapping: Integrated software for genetic linkage map construction and quantitative trait locus mapping in biparental populations. Crop Journal 3:269-283. https://doi.org/10.1016/j.cj.2015.01.001
Mistry J, Chuguransky S, Williams L, Qureshi M, Salazar G A, Sonnhammer E L L, Tosatto S C E, Paladin L, Raj S, Richardson L J, Finn R D, Bateman A, 2020. Pfam: The protein families database in 2021. https://doi.org/10.1093/nar/gkaa913
Pageau D, Lajeunesse J, Lafond J, 2006. Impact de I'hernie des crucifères [Plasmodiophora brassicae] sur la productivité et la qualité du canola. Canadian Journal of Plant Pathology 28:137-143. doi:10.1080/07060660609507280.
Pandey M K, Khan A W, Singh V K, Vishwakarma M K, Shasidhar Y, Kumar V, Garg V, Bhat R S, Chitikineni A, Janila P, Guo B, Varshney R K, 2017. QTL-seq approach identified genomic regions and diagnostic markers for rust and late leaf spot resistance in groundnut (Arachis hypogaea L.). Plant Biotechnology Journal 15:927-941. https://doi.org/10.1111/pbi.12686
Pertea M, Pertea G, Antonescu C, Chang T-C, Mendell J T, Salzberg S L, 2015. StringTie enables improved reconstruction of a transcriptome from RNA-seq reads. Nat Biotechnol 33:290-295. https://doi.org/10.1038/nbt.3122
Pilet-Nayel M L, Moury B, Caffier V, Montarry J, Kerlan M C, Fournet S et al., 2017. Quantitative resistance to plant pathogens in pyramiding strategies for durable crop protection. Frontiers in Plant Science 8:1838. doi:10.3389/fpls.2017.01838. PMID:29163575.
Poudel R S, Al-Hashel A F, Gross T, Gross P, Brueggeman R, 2018. Pyramiding rpg4- and Rpg1-mediated stem rust resistance in barley requires the Rrr1 gene for both to function. Frontiers in Plant Science 9:1789. doi:10.3389/fpls.2018.01789. PMID:30568667.
Rahman H, Peng G, Yu F, Falk K C, Kulkarni M, Selvaraj G, 2014. Genetics and breeding for clubroot resistance in Canadian spring canola (Brassica napus L.). Canadian Journal of Plant Pathology 36:122-134. DOI:10.1080/07060661.2013.862571
Rahman H, Shakir A, Hasan M J, 2011. Breeding for clubroot resistant spring canola (Brassica napus L.) for the Canadian prairies: can the European winter canola cv. Mendel be used as a source of resistance? Canadian Journal of Plant Science 91:447-458. DOI:10.4141/cjps10073
Sakamoto K, Saito A, Hayashida N, Taguchi G, Matsumoto E, 2008. Mapping of isolate-specific QTLs for clubroot resistance in Chinese cabbage (Brassica rapa L. ssp. pekinensis). Theoretical and Applied Genetics 117:759-767. doi:10.1007/s00122-008-0817-0. PMID:18612625.
Shah N, Sun J, Yu S, Yang Z, Wang Z, Huang F et al., 2019. Genetic variation analysis of field isolates of clubroot and their responses to Brassica napus lines containing resistant genes CRb and PbBa8.1 and their combination in homozygous and heterozygous state. Molecular Breeding 39:153. doi:10.1007/s11032-019-1075-3
Song J M, Guan Z, Hu J, Guo C, Yang Z, Wang S, Liu D, Wang B, Lu S, Zhou R, 2020. Eight high-quality genomes reveal pan-genome architecture and ecotype differentiation of Brassica napus. Nature Plants 6(1):34-45. https://doi.org/10.1038/s41477-019-0577-7
Strelkov S E, Hwang S F, Manolii V P, Cao T S, Feindel D, 2016. Emergence of new virulence phenotypes of Plasmodiophora brassicae on canola (Brassica napus) in Alberta. Canada. European Journal of Plant Pathology 145:517-529. DOI:10.1007/s10658-016-0888-8.
Strelkov S E, Hwang S F, Manolii V P, Cao T, Fredua-Agyeman R, Harding M W, Peng G, Gossen B D, McDonald M R, Feindel D, 2018. Virulence and pathotype classification of Plasmodiophora brassicae populations collected from clubroot resistant canola (Brassica napus) in Canada. Canadian Journal of Plant Pathology 40:284-298. DOI:10.1080/07060661.2018.1459851.
Strelkov S E, Hwang S F, Manolii V P, Turnbull G, Fredua-Agyeman R, Hollman K, Kaus S, 2021. Characterization of clubroot (Plasmodiophora brassicae) from canola (Brassica napus) in the Peace Country of Alberta, Canada. Canadian Journal of Plant Pathology 43:155-161. DOI:10.1080/07060661.2020.1776931.
Strelkov S E, Tewari J P, Smith-Degenhardt E, 2006. Characterization of Plasmodiophora brassicae populations from Alberta, Canada. Canadian Journal of Plant Pathology 28:467-474. https://doi.org/10.1080/07060660609507321.
Summanwar A, Basu U, Rahman H, Kav N, 2019. Identification of lncRNAs responsive to infection by Plasmodiophora brassicae in clubroot-susceptible and -resistant Brassica napus lines carrying resistance introgressed from rutabaga. Mol Plant Microbe Interact 32(10):1360-1377. https://doi.org/10.1094/MPMI-12-18-0341-R
Summanwar A, Basu U, Kav N N V, Rahman H, 2020. Identification of lncRNAs in response to infection by Plasmodiophora brassicae in Brassica napus and development of lncRNA-based SSR markers. Genome 64(5):547-566. https://doi.org/10.1139/gen-2020-0062
Tewari J P, Strelkov S E, Orchard D, Hartman M, Lange R M, Turkington T K, 2005. Identification of clubroot of crucifers on canola (Brassica napus) in Alberta. Canadian Journal of Plant Pathology 27:143-144. DOI:10.1080/07060660509507206.
Tomita H, Shimizu M, Asad-Ud Doullah M, Fujimoto R, Okazaki K, 2013. Accumulation of quantitative trait loci conferring broad-spectrum clubroot resistance in Brassica oleracea. Molecular Breeding 32:889-900. doi:10.1007/s11032-013-9918-9
Uhrig R G, Schläpfer P, Roschitzki B, Hirsch-Hoffmann M, Gruissem W, 2019. Diurnal changes in concerted plant protein phosphorylation and acetylation in Arabidopsis organs and seedlings. Plant J 99(1):176-194. https://doi.org/10.1111/tpj.14315
Untergasser A, Cutcutache I, Koressaar T, Ye J, Faircloth B C, Remm M, Rozen S G, 2012. Primer3-new capabilities and interfaces. Nucleic Acids Res 40(15):e115. DOI: 10.1093/nar/gks596
Van Ooijen J, 2011. Multipoint maximum likelihood mapping in a full-sib family of an outbreeding species. Genetics Research 93:343-349. doi: 10.1017/S0016672311000279
Voorrips R E, Visser D L, 1993. Examination of resistance to clubroot in accessions of Brassica oleracea using a glasshouse seedling test. Netherlands Journal of Plant Pathology 99:269-276. https://doi.org/10.1007/BF01974308
Wallenhammar A-C, 1996. Prevalence of Plasmodiophora brassicae in a spring oilseed rape growing area in central Sweden and factors influencing soil infestation levels. Plant Pathology 45:710-719. doi:10.1046/j.1365-3059.1996.d01-173.x.
Wang X, et al., 2011. The genome of the mesopolyploid crop species Brassica rapa. Nature Genetics 43:1035-1039.
Wang Z, Megha S, Kebede B, Kav N N, Rahman H, 2022. Genetic and molecular analysis reveals that two major loci and their interaction confer clubroot resistance in canola introgressed from rutabaga. The Plant Genome, e20241. https://doi.org/10.1002/tpg2.20241.
Werner S, Diederichsen E, Frauen M, Schondelmaier J, Jung C, 2008. Genetic mapping of clubroot resistance genes in oilseed rape. Theor Appl Genet 116:363-372. DOI 10.1007/s00122-007-0674-2
Werner K, Friedt W, Ordon F, 2005. Strategies for pyramiding resistance genes against the barley yellow mosaic virus complex (BaMMV, BaYMV, BaYMV-2). Molecular Breeding 16:45-55. doi:10.1007/s11032-005-3445-2
You F M, Huo N, Gu Y, Luo M-C, Ma Y, Hane D, Lazo G R, Dvorak J, Anderson O D, 2008. BatchPrimer3: A high through-put web application for PCR and sequencing primer design. BMC Bioinformatics 9:253. https://doi.org/10.1186/1471-2105-9-253
Yu F, Zhang X, Peng G, Falk K C, Strelkov S E, Gossen B D, 2017. Genotyping-by-sequencing reveals three QTL for clubroot resistance to six pathotypes of Plasmodiophora brassicae in Brassica rapa. Scientific Reports 7:4516. doi:10.1038/s41598-017-04903-2. PMID:28674416.
Yu G, Wang L, Han Y, He Q, 2012. ClusterProfiler: an R package for comparing biological themes among gene clusters. OMICS 16(5):284-287. https://doi.org/10.1089/omi.2011.0118
Zhang L, Cai X, Wu J, Liu M, Grob S, Cheng F, Liang J, Cai C, Liu Z, Liu B, Wang F, Li S, Liu F, Li X, Cheng L, Yang W, Li M-H, Grossniklaus U, Zheng H, Wang X, 2018. Improved Brassica rapa reference genome by single-molecule sequencing and chromosome conformation capture technologies. Horticulture Research 5:50. doi: 10.1038/s41438-018-0071-9

All publications, patents and patent applications mentioned in this Specification are indicative of the level of skill those skilled in the art to which this invention pertains and are herein incorporated by reference to the same extent as if each individual publication patent, or patent application was specifically and individually indicated to be incorporated by reference.

The invention being thus described, it will be obvious that the same may be varied in many ways. Such variations are not to be regarded as a departure from the spirit and scope of the invention, and all such modification as would be obvious to one skilled in the art are intended to be included within the scope of the following claims.

1. An isolated Plasmodiophora brassicae (P. brassicae) resistant Brassica plant cell comprising in its genome an A09 resistance locus comprising the interval from about 28.7 to about 41.5 Mb of A09.

2. The P. brassicae resistant Brassica plant cell of claim 1, wherein the A09 resistance locus is in the interval from about 38.01-41.5 Mb of A09.

3. The P. brassicae resistant Brassica plant cell of claim 1, wherein the A09 resistance locus is in the interval from about 28.7-37.3 Mb of A09.

4. The P. brassicae resistant Brassica plant cell of claim 1, wherein the A09 resistance locus is at about 28.7-37.3 Mb of A09.

5. The P. brassicae resistant Brassica plant cell of claim 1, further comprising in its genome one or more of:
an A03 resistance locus comprising the interval from about 1.7 to about 14.5 Mb of A03,
an A02 resistance locus comprising the interval from about 1 to about 4 Mb of A02, or
an A08 resistance locus comprising the interval from about 14.34 Mb to about 20.87 Mb of A08.

6. The P. brassicae resistant Brassica plant cell of claim 5, wherein the A03 resistance locus is in the interval of about 1.7-10.2 Mb of A03.

7. The P. brassicae resistant Brassica plant cell of claim 5, wherein the A03 resistance locus is in the interval of about −10.9-14.5 Mb of A03.

8. The P. brassicae resistant Brassica plant cell of claim 1, wherein said plant cell is heterozygous for said resistance locus or homozygous for said resistance locus.

9. (canceled)

10. The P. brassicae resistant Brassica plant cell of claim 1, wherein said plant cell is resistant to one or more of to one or more of P. brassicae pathotypes 3H, 2B, 3A, 3D, 5G, 5X, 5L, 5O, 8E, or 8P.

11. The P. brassicae resistant Brassica plant cell of claim 1, wherein the Brassica plant cell is Brassica napus, Brassica rapa, or Brassica oleracea.

12. A Seeds, fruit and/or plant part of comprising a Brassica plant cell as claimed in claim 1.

13. A method for producing a clubroot resistant Brassica plant, said method comprising:
identifying at least one Brassica plant comprising in its genome an A09 resistance locus comprising the interval from about 28.7 to about 41.5 Mb of A09, and
selecting a plant comprising said A09 resistance locus comprising the interval from about 28.7 to about 41.5 Mb of A09.

14. The method of claim 13, wherein the A09 resistance locus is in the interval from about 38.01-41.5 Mb of A09.

15. The method of claim 13, wherein the A09 resistance locus is in the interval from about 28.7-37.3 Mb of A09.

16. The method of claim 13, wherein the A09 resistance locus is at about 28.7-37.3 Mb of A09.

17. The method of claim 13, further comprising identifying in its genome one or more of:
an A03 resistance locus comprising the interval from about 1.7 to about 14.5 Mb of A03,
an A02 resistance locus comprising the interval from about 1 to about 4 Mb of A02, or
an A08 resistance locus comprising the interval from about 14.34 Mb to about 20.87 Mb of A08, and
selecting a plant comprising said one or more resistance locus.

18. The method of claim 17, wherein the A03 resistance locus is in the interval of about 1.7-10.2 Mb of A03.

19. The method of claim 17, wherein the A03 resistance locus is in the interval of about −10.9-14.5 Mb of A03.

20. The method of claim 13, wherein said plant is heterozygous for said resistance locus or homozygous for said resistance locus.

21. (canceled)

22. The method of claim 13, wherein said plant is resistant to one or more of to one or more of P. brassicae pathotypes 3H, 2B, 3A, 3D, 5G, 5X, 5L, 5O, 8E, or 8P.

23-34. (canceled)


SEQUENCE LISTING (1 sequence)

SEQ ID NO: 1 - DNA, 19 bp, synthetic construct
  source 1..19: mol_type = other DNA
cacgacgttgtaaaacgac